(19) United States
(12) Patent Application Publication
(10) Pub. No.: US 2021/0071180 A1
(43) Pub. Date: Mar. 11, 2021

(54) MICRORNA 584-5P COMPOSITIONS AND METHODS FOR TREATING CANCER

(71) Applicant: BOARD OF REGENTS, THE UNIVERSY OF TEXAS SYSTEM, Austin, TX (US)

(72) Inventors: Manjeet K. Rao, San Antonio, TX (US); Nourhan Abdelfattah, San Antonio, TX (US)

(21) Appl. No.: 17/049,836

(22) PCT Filed: Apr. 22, 2019
(86) PCT No.: PCT/US2019/028497

§ 371 Date: Oct. 22, 2020

Related U.S. Application Data
(60) Provisional application No. 62/661,300, filed on Apr. 23, 2018.

(51) Int. Cl. C12N 15/113; A61K 31/7105; A61K 31/475; A61K 31/337; A61P 35/00

(52) U.S. Cl. CPC C12N 15/113; A61K 31/7105; A61K 31/475; A61N 5/10; A61P 35/00; C12N 2310/141; C12N 2320/31; A61K 31/337

(57) ABSTRACT

The disclosure relates to compositions and methods of treating medulloblastoma in a subject. The method also comprises administering to a subject in need of treatment an effective amount of miR-584-5p.

### CROSS REFERENCE TO RELATED APPLICATIONS

This application claims the benefit of the filing date of U.S. Provisional Application 62/661,300, which was filed on Apr. 23, 2018. The content of this earlier filed application is hereby incorporated by reference herein in its entirety.

### INCORPORATION OF THE SEQUENCE LISTING

The present application contains a sequence listing that was submitted in ASCII format via EFS-Web concurrent with the filing of the application, containing the file name “21105_03056P1_SL.txt” which is 4,096 bytes in size, created on Apr. 16, 2019, and is herein incorporated by reference in its entirety.

### BACKGROUND

Medulloblastoma (MB) is the most common malignant pediatric brain cancer, which accounts for >10% of childhood cancer death (Pizer, B. L. & Clifford, S. C. Br J Neurosurg 23, 364-375, (2009); and Hill, R. M. et al. Cancer Cell 27, 72-84, (2015)). MB is classified into four molecular subtypes: WNT, Sonic Hedgehog, group 3, and group 4 (Northcott, P. A., et al. Expert Rev Neurother 12, 871-884, (2012); and Taylor, M. D. et al. Acta Neuropathol 123, 465-472, (2012)). Group 3 has the worst prognostic outcome, with 40%-45% of patients presenting with metastatic lesions (Northcott, P. A. et al. J Clin Oncol 29, 1408-1414, (2011)). MB is deemed high risk when the tumor is metastatic, has large cell/anaplastic phenotype, or is c-Myc amplified (Ellison, D. W. et al. J Clin Oncol 29, 1400-1407, (2011)). Despite the progress made in treating MB, the 5-year survival rate for high-risk tumors remains poor and risk of recurrence within 2 years of treatment is still high (Kool, M. et al. PLoS One 3, (2008)). Because of the highly toxic side effects of radiation and chemotherapy, surviving children have reduced quality of life. For example, craniospinal exposure of 35.5 Gy followed by up to 54-Gy radiation boosts to the posterior fossa leads to severe neurocognitive deficits, chronic neuropathy, and chronic hypopituitarism as well as secondary malignancies (Rieken, S. et al. Int J Radiat Oncol Biol Phys 81, (2011)). Similarly, vincristine (VCR)—a microtubule-interfering chemotherapeutic agent routinely administered as a radiotherapy adjuvant to both high- and average-risk MB patients (Packer, R. J. et al. J Neurosurg 81, 690-698, (1994); and Kim, H. et al. Childs Nery Syst 29, 1851-1858, (2013))—causes cumulative dose-dependent neurotoxicity that includes, but is not limited to, sensorimotor and autonomic neuropathy, hearing loss, mononeuropathy, and seizures (Silvani, A. et al. J Neurooncol 106, 595-600, (2012)). Therefore new targets that can serve as more effective and less toxic therapeutics to treat MB are needed.

### SUMMARY

Disclosed herein are methods of increasing sensitivity of one or more medulloblastoma cancer cells to radiotherapy or chemotherapy, the method comprising: (a) identifying a subject in need of treatment; and (b) administering to the subject a therapeutically effective amount of a miR-584-5p before, during or after administration of radiotherapy or a chemotherapeutic agent, in an amount sufficient to increase sensitivity of one or more medulloblastoma cancer cells to the radiotherapy or chemotherapeutic agent.

Disclosed herein are synergistic compositions for the treatment of cancer or tumor expressing increased levels of HDAC1 or eIF4E3, the compositions comprising a miR-584-5p, and a microtubule-interfering chemotherapeutic agent.

Disclosed herein are methods of treating medulloblastoma in a subject, the methods comprising: (a) identifying a subject in need of treatment; and (b) administering to the subject a therapeutically effective amount of a miR-584-5p.

Disclosed herein are methods of reducing growth of medulloblastoma cancer cells, the methods comprising: (a) administering a therapeutically effective amount of a composition comprising miR-584-5p to a subject having or suspected of having cancer cells reduced in expression of miR-584-5p; and (b) administering to the subject a therapeutically effective amount of vincristine before, after or during administration of the composition comprising miR-584-5p, and wherein the medulloblastoma cancer cells have become sensitized to vincristine.

Disclosed herein are methods of suppressing expression of a target gene in a cell, the methods comprising contacting a cell with miR-584-5p, wherein the target gene encodes HDAC1 or eIF4E3; thereby suppressing the expression of the target gene in cell when compared to a reference sample.

Disclosed herein are methods of comprising the steps in the following order: (a) obtaining a sample comprising tumor cells from a cancer patient; (b) measuring a level of miR-584-5p in one or more of the tumor cells of the sample; (c) identifying the cancer patient as a suitable candidate for treatment with a composition comprising miR-584-5p if the level of miR-584-5p is lower than a level of miR-584-5p in a control sample and identifying the cancer patient as an unsuitable candidate for treatment with a composition comprising miR-584-5p if the level of miR-584-5p is higher than a level of miR-584-5p in a control sample; and (d) administering the composition comprising miR-584-5p to the cancer patient identified as the suitable candidate, and not administering the composition comprising miR-584-5p to the cancer patient identified as the unsuitable candidate.

Disclosed herein are methods of inhibiting medulloblastoma growth, the methods comprising: (a) identifying a subject in need of treatment; and (b) administering to the subject a therapeutically effective amount of a miR-584-5p before, during or after administration of radiotherapy and/or a chemotherapeutic agent, in an amount sufficient to inhibit medulloblastoma growth.

Disclosed herein are methods of enhancing the efficacy of radiotherapy and/or a chemotherapeutic agent in a subject with medulloblastoma, the methods comprising administering to the subject: (a) an effective amount of a chemotherapeutic agent and/or radiotherapy; and (b) a therapeutically effective amount of a miR-584-5p, wherein the administration of the miR-584-5p enhances the efficacy of the radiotherapy and/or the chemotherapeutic agent in the subject with medulloblastoma.

Other features and advantages of the present compositions and methods are illustrated in the description below, the drawings, and the claims.

### BRIEF DESCRIPTION OF THE DRAWINGS

FIGS. 1A-H show that the high-throughput functional screen identified miR-584-5p as a new therapeutic adjuvant for improving the efficacy of vincristine (VCR) in MB. FIG. 1A shows an outline of the primary screen and list of drug-sensitizer, drug-desensitizer, and drug-neutral miRNAs. A total of 1,902 miRNA mimics arrayed in 96-well plates were screened in triplicate. (B) Distribution of z-scores for cells treated with vehicle (blue dots) or VCR (red dots). Cells transfected with negative control (NC) miRNAs (ath-miR-416 or cel-miR-243) are represented by gray dots. FIGS. 1C-F are dose-response curves showing VCR sensitivity in miRNA negative control (NC) (blue line) and miR-584-5p mimic-transfected D556Med, D458Med, D425Med, and DAOY cells. FIGS. 11 G-H show the synergistic effect of miR-584-5p with VCR. D556Med cells were treated with increasing concentrations of miR-584-5p and VCR before being subjected to cell viability assay using alamarBlue cell viability assay.

FIGS. 2A-D validate miR-585-p as a therapeutic adjuvant for improving the efficacy of vincristine (VCR) in MB. FIG. 2A shows the viability of D556Med, D425Med, D458Med, and DAOY cells treated with increasing concentrations of Vincristine (VCR). FIG. 2B shows the CellTiter-Glo raw signal of primary screen plate controls in vehicle vs. VCR plates confirming 20% reduction in viability (IC₂₀). FIG. 2C represents the quality control analysis showing normal distribution of CellTiter-Glo raw signal of primary screen plate controls in vehicle vs. VCR plates. FIG. 2D shows the relative viability of D556Med cells transfected with 50 nM miR-NC or miR-584-5p mimic for 24 hours, followed by treatment with 5 nM VCR or vehicle for 72 hours. Cell viability was assessed using alamar Blue cell viability assay. The p-value was calculated using one-way ANOVA followed by Sidak's multiple comparisons test. Error bars represent mean±SEM. ****, p<0.0001; ***, p<0.001.

FIGS. 3A-G show that MiR-584-5p acts as a tumor suppressor and sensitizes VCR response in vivo. FIG. 3A shows cell proliferation in negative control miRNA (miR-NC) and miR-584-5p mimic-transfected MB cells. FIG. 3B is photomicrograph that shows changes in cell morphology of D556Med cells transfected with miR-NC or miR-584-5p mimic. Line graph shows cell proliferation of D556Med cells transfected with miR-NC or miR-584-5p mimic. FIG. 3C shows the results of a clonogenic assay of D556Med cells transfected with miR-NC or miR-584-5p mimic. FIG. 3D is a photomicrograph that shows number of D556Med cells transfected with miR-NC or miR-584-5p mimic. FIG. 3E is a line graph that shows tumor growth in athymic nude mice intracranially injected with DAOY-GFP-Luciferase cells transfected with miR-NC or miR-584-5p mimic and treated with vehicle control (dimethyl sulfoxide [DMSO]; n=12/group) or vincristine (VCR; n=6/group). FIG. 3F shows live bioluminescence (BLI) images of mice intracranially injected with DAOY-GFP-Luciferase cells transfected with miR-NC or miR-584-5p mimic and treated with vehicle or VCR. FIG. 3G shows the Kaplan-Meier survival curve of mice treated with miR-NC or miR-584-5p mimic (n=10/group).

FIGS. 4A-F show that miR-584-5p reduced the short-term and long-term viability as well as migration of MB cells. Photomicrograph showing cell number and morphology of D458Med (A) or D425Med (B) cells transfected with miR-NC or miR-584-5p. FIG. 4C shows the results of a clonogenic assay of DAOY cells transfected with miR-NC or miR-584-5p mimic. FIG. 4D shows animal weights at week 6. FIG. 4E shows the Taqman qPCR analysis of miR-584-5p expression in induced pluripotent stem cell-derived neural progenitor cells 48 hours after transfection with 50 nM miR-NC or miR-584-5p mimic. FIG. 4F shows cell proliferation of D556Med cells transfected with miR-NC or miR-584-5p mimic.

FIGS. 5A-E show that MiR-584-5p targets cell cycle, cancer, microtubule dynamics, and translation-associated genes. FIG. 4A shows the hierarchical clustering of gene expression changes in D425Med and D458Med cells treated with miR-NC or miR-584-5p mimic for 48 hours. FIG. 5B demonstrates the ingenuity pathway analysis showing enriched biological pathways in miR-584-5p mimic-treated MB cells. FIG. 5C shows luciferase-eIF4E3/HDAC1 3′-UTR constructs (left); eIF4E-3′UTR: wild type 3′ UTR (SEQ ID NO: 4); miR-584-5p (SEQ ID NO: 1); seed sequence mutant (SEQ ID NO: 5); and binding site mutant (SEQ ID NO: 6); and HDAC1-3′UTR: wild type 3′ UTR (SEQ ID NO: 7); miR-584-5p (SEQ ID NO: 1); seed sequence mutant (SEQ ID NO: 8); and binding site mutant (SEQ ID NO: 9); FIG. 5D shows the qPCR analysis of eIF4E3 and HDAC1 expression in MB cells (D556Med, D425Med, D458Med, and DAOY) transfected with miR-NC or miR-584-5p mimic. FIG. 5E shows the Western blot analysis of target genes in miR-NC or miR-584-5p mimic-transfected D556Med, D425Med, D458Med, and DAOY cells.

FIGS. 6A-H show that eIF4E3 and HDAC1 promote growth and progression as well as inhibit vincristine (VCR) sensitivity of MB cells. FIG. 6A shows proliferation of D556Med cell transfected with scramble- or eIF4E3/HDAC1-siRNA. FIG. 6B shows the difference in morphology between D556Med cells transfected with scramble-siRNA, eIF4E3-siRNA, or HDAC1-siRNA. FIG. 6C shows the results of a colony-forming assay of D556Med cells transfected with scramble-siRNA, eIF4E3-siRNA, or HDAC1-siRNA. FIG. 6D shows migrated D556Med cells transfected with scramble-siRNA, eIF4E3-siRNA, or HDAC1-siRNA. FIG. 6E shows the mean tumor volume in athymic nude mice intracranially injected with DAOY-GFP-luciferase cells transfected with scramble-siRNA or eIF4E3-siRNA or HDAC1-siRNA (n=6 mice/group). FIG. 6F shows the live bioluminescence (BLI) images of mice injected with DAOY-GFP-luciferase cells transfected with scramble-siRNA or eIF4E3-siRNA or HDAC1-siRNA (n=6 mice/group). FIG. 6G shows the VCR dose-response curve of D556Med cells transfected with scramble-siRNA or eIF4E3-siRNA or HDAC1-siRNA and treated with VCR or vehicle. FIG. 6H shows that miR-584-5p rescues cancer growth-promoting effects of eIF4E3 and HDAC1.

FIGS. 7A-D shows that Silencing HDAC1 or eIF4E3 led to significantly reduced viability, proliferation, and migration of MB cell. FIG. 7A shows a volcano plot of gene expression changes in D425Med and D458Med cells treated with miR-NC or miR-584-5p mimic for 48 hours. Red dots represent target genes. FIG. 7B shows the relative viability of D556Med, D425Med, D458Med, and DAOY cells transfected with 50 nM Scramble, eIF4E3, or HDAC1 siRNAs. FIG. 7C shows the representative images of CyQuant cell proliferation nuclear staining. FIG. 7D shows that MiR-584-5p rescues cancer migration-promoting effects of eIF4E3 and HDAC1.

FIGS. 8A-D show that MiR-584-5p-HDAC1/eIF4E3 signaling axis regulates cell cycle progression of MB cells. FIG. 8A shows the FACS analysis of cell cycle progression in D556Med cells transfected with miR-NC or miR-584-5p and treated with vehicle or vincristine (VCR). FIG. 8B shows the FACS analysis of Annexin V-FITC-positive cells in D556Med cells transfected with miR-NC or miR-584-5p and treated with vehicle or VCR. FIG. 8C shows the FACS analysis of cell cycle progression in D556Med cells transfected with scramble-siRNA or eIF4E3-siRNA or HDAC1-siRNA. FIG. 8D shows the FACS analysis of Annexin V-FITC-positive cells in D556Med cells transfected with scramble-siRNA or eIF4E3-siRNA or HDAC1-siRNA.

FIGS. 9A-C shows that MiR-584-5p-HDAC1/eIF4E3 signaling axis regulates cell cycle progression of MB cells. FIG. 9A shows the FACS analysis of cell cycle progression in D425Med, D458Med, and DAOY cells transfected with miR-NC or miR-584-5p and treated with vehicle or vincristine (VCR). FIG. 9B shows the microscopic count of mitotic DAOY cells transfected with miRNC or miR-584-5p and treated with vehicle or VCR. FIG. 9C shows the FACS analysis of Annexin V-FITC positive cells in D425Med, D458Med, and DAOY cells transfected with miR-NC or miR-584-5p and treated with vehicle or VCR.

FIGS. 10A-C show that PGC-1α expression is increased in miR-584-5p-treated MB cells. FIG. 10A shows the Western blot analysis of eIF4E1 in miR-584-5p-overexpressing, target genes depleted or overexpressing D556Med cells. FIG. 10B shows the results of qRT-PCR showing PGC-1a expression levels in miR-584-5p mimic transfected D556Med, D425Med, D458Med cells. FIG. 10C shows PGC-1α expression levels in human medulloblastoma samples.

FIGS. 11A-H show that MiR-584-5p-eIF4E3/HDAC1 signaling axis regulates microtubule dynamics in MB cells. FIG. 11A shows the results of qPCR analysis of TUBB4a in D556Med, D425Med, D458Med, and DAOY cells transfected with miR-NC or miR-584-5p mimic. FIG. 11B shows the results of qPCR analysis of β-tubulin isotypes in D556Med cells transfected with miR-NC or miR-584-5p mimic. FIG. 11C shows types of spindle defects (above) immunofluorescence images of defective mitotic spindles in D556Med cells transfected with 50 nM miR-NC or miR-584-5p mimic. DNA is stained with DAPI (blue), whereas spindle is stained with β-tubulin (red) (below). FIG. 11D shows immunofluorescence images of defective mitotic spindles in D556Med cells transfected with scramble-siRNA or eIF4E3-siRNA or HDAC1-siRNA. FIG. 11E shows a cell undergoing mitotic catastrophe (above) and immunofluorescence images of miR-584-5p-transfected D556Med cells undergoing mitotic catastrophe (below). FIG. 11F shows the number of miR-NC or miR-584-5p mimic-transfected D556Med cells undergoing mitotic catastrophe and with defective spindles. FIG. 11G shows cells undergoing aneuploidy and cytokinesis failure (above) and immunofluorescence images of miR-584-5p mimic-transfected D556Med cells undergoing cytokinesis failure and exhibiting aneuploidy (below). FIG. 11H shows the number of D556Med cells transfected with miR-NC or miR-584-5p mimic undergoing aneuploidy and cytokinesis failure.

FIGS. 12A-B show miR-584-5p affect microtubules dynamics. FIG. 12A shows the results of qPCR analysis of TUBB3 in D556Med, D425Med, D458Med, and DAOY cells transfected with miR-NC or miR-584-5p mimic. FIG. 12B shows immunofluorescence images of defective mitotic spindles in DAOY cells transfected with 50 nM miR-NC or miR-584-5p mimic in the presence and absence of VCR. DNA is stained with DAPI (blue), whereas spindle is stained with b-tubulin (green).

FIGS. 13A-E shows that MiR-584-5p damages DNA and sensitizes radiation response in MB cells. FIG. 13A shows IR dose-response curves of D556Med, D458Med, D425Med, and DAOY cells transfected with miR-NC or miR-584-5p mimic and treated with increasing dose of IR. FIG. 13B shows ionizing radiation (IR) dose-response curves of D556Med cells transfected with scramble-siRNA or eIF4E3-siRNA or HDAC-siRNA. FIG. 13C-D show representative immunofluorescence images showing 53BP1 foci in D556Med cells transfected with miR-NC or miR-584-5p mimic (C) or scramble-siRNA or eIF4E3/HDAC1-siRNAs (D) and treated with or without 10 Gy of IR. FIG. 13E shows the results of a DR-GFP reporter assay (left) and flow cytometry analysis showing number of GFP-positive cells reflecting homologous recombination (HR) events (right). FIG. 13F shows the results of a Western blot analysis of D556Med cells treated with miR-NC or miR-584-5p mimic using antibodies against indicated proteins.

FIGS. 14A-G shows that MiR-584-5p inhibits MB stem cell proliferation and regulates MYC expression. FIG. 14A shows medullospheres grown from D556Med cells transfected with miR-NC or miR-584-5p mimic. FIG. 14B shows medullospheres grown from D556Med cells transfected with scramble-siRNA or eIF4E3/HDAC1-siRNAs. FIG. 14C shows qPCR analysis of stem cell markers in D556Med cells transfected with miR-NC or miR-584-5p mimic FIG. 14D shows qPCR analysis of MYC expression in D556Med, D425Med, D458Med, and DAOY cell lines. FIG. 14E shows the results of a Western blot analysis of c-Myc in D556Med, D425Med, and D458Med cells transfected with miR-NC or miR-584-5p mimic. FIG. 14F shows the results of a Western blot analysis of MYC and eIF4E in D556Med cells transfected with siRNA against scramble, eIF4E3, or c-Myc. FIG. 14G shows that miR-584-5p and eIF4E3 rescue c-Myc-dependent MB cell growth.

FIGS. 15A-C shows that miR-584-5p rescues cancer growth-promoting effects of c-Myc. FIG. 15A depicts a photomicrograph showing migrated MB cells transfected with control plasmid, c-Myc overexpression plasmid or c-Myc overexpression plasmid+miR-584-5p mimic. FIG. 15B shows the number of colonies obtained from clonogenic assay in MB cells transfected with control plasmid, c-Myc overexpression plasmid or c-Myc overexpression plasmid+miR-584-5p mimic. FIG. 15C shows that eIF4E3 depletion rescues cancer growth-promoting effects of MYC.

FIGS. 16A-D shows that miR-584-5p expression is significantly lower in pediatric MB patient samples, whereas eIF4E3 and HDAC1 are highly expressed. (A) miR-584-5p expression levels in human MB samples. FIG. 16B shows eIF4E3 and HDAC1 expression levels in human MB samples. FIG. 16C shows the results of Taqman qPCR analysis of miR-584 expression in iPSC-derived mature neurons, neural progenitor cells (NPCs), and MB patient derived xenografts (PDXs; n=6). FIG. 16D depicts a model showing miR-584-5p mechanisms of action. ****, p<0.0001; ***, p<0.001; **, p<0.01.

FIGS. 17A-C shows miR-584-5p conjugated to nanoparticles. FIG. 17A is a schematic of ligand/antibody conjugated to a PLGA-Nanoparticle (PLGA-NP) loaded with miRNA/drug. FIG. 17B is a transmission electron microscopy (TEM) image showing shape and size of PLGA-NP. FIG. 17C shows the results of an evaluation of cy5-labeled miR-584-5p loaded PLGA NPs internalized into DAOY cells. DAOY cells (cultured on cover slips) transfected with either 50 ng of Cy5-labled-miR-584-5p-PLGA for 24 hours before being fixed with 4% paraformaldehyde and mounted with DAPI and actin antibody. The arrows indicate the uptake of Cy5-miR-584-5p-PLGA by DAOY cells Immunofluorescence was performed on Nikon-Eclipse-2000 microscope.

FIG. 18 shows systemic delivery of miR-584-5p mimic inhibits medulloblastoma growth. Tumor volume was assessed starting from before miR-584-5p delivery and until mice were sacrificed at day 24. Using ROI analysis, tumor light intensity was calculated in photons/s, which corresponds with the number of live cells. DAOY-Luc cells were intracranially implanted and when tumor reached measurable size, miR-584-5p conjugated with lipid nanoparticle (MaxSuppressor In vivo LANCEr II, Biooscientific Inc) was delivered via tail vein injection.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, the subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step.

As used herein, the term “treating” or “treatment” refers to partially or completely alleviating, ameliorating, relieving, delaying onset of, inhibiting or slowing progression of, reducing severity of, and/or reducing incidence of one or more symptoms or features of, or otherwise prevent, hinder, retard, or reverse the progression of a particular disease, disorder, and/or condition or other undesirable symptom(s). Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be cancer.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, malady, disorder, sickness, illness, complaint, affection.

As used herein, the term “normal” refers to an individual, a sample or a subject that does not have cancer or does not have medulloblastoma.

The terms “vector” or “construct” refer to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “expression vector” is herein to refer to vectors that are capable of directing the expression of genes to which they are operatively-linked. Common expression vectors of utility in recombinant DNA techniques are often in the form of plasmids. Recombinant expression vectors can comprise a nucleic acid as disclosed herein in a form suitable for expression of the acid in a host cell. In other words, the recombinant expression vectors can include one or more regulatory elements or promoters, which can be selected based on the host cells used for expression that is operatively linked to the nucleic acid sequence to be expressed.

As used herein, the term “sensitivity” refers to the ability of a cell to survive exposure to an agent designed to inhibit the growth of the cell, kill the cell or inhibit one or more cellular functions.

As used herein, the term “synergistic composition” refers to the application of the combination of miR-584-5p (or a precursor thereof) and an additional therapeutic agent. The synergistically effective amount refers to the amount of each component which, in combination, is effective in inhibiting growth, or reducing viability, of cancer cells, and which produces a response greater than either component alone.

“Modulate”, “modulating” and “modulation” as used herein mean a change in activity or function or number. The change may be an increase or a decrease, an enhancement or an inhibition of the activity, function or number.

The terms “alter” or “modulate” can be used interchangeable herein referring, for example, to the expression of a nucleotide sequence in a cell means that the level of expression of the nucleotide sequence in a cell after applying a method as described herein is different from its expression in the cell before applying the method.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in an aspect, the increase or promotion can be a 10, 20, 30, 40, 50, 60, 70, 80, 90, 100%, or more, or any amount of promotion in between compared to native or control levels. In an aspect, the increase or promotion is 10-20, 20-30, 30-40, 40-50, 50-60, 60-70, 70-80, 80-90, or 90-100% as compared to native or control levels. In an aspect, the increase or promotion is 0-25, 25-50, 50-75, or 75-100%, or more, such as 200, 300, 500, or 1000% more as compared to native or control levels. In an aspect, the increase or promotion can be greater than 100 percent as compared to native or control levels, such as 100, 150, 200, 250, 300, 350, 400, 450, 500% or more as compared to the native or control levels. As used herein, promoting can also mean enhancing.

As used herein, the term “inhibit” or “inhibiting” mean decreasing tumor cell growth rate from the rate that would occur without treatment and/or causing tumor mass (e.g., cancer) to decrease. Inhibiting also include causing a complete regression of the tumor (e.g., cancer).

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, certain changes and modifications may be practiced within the scope of the appended claims.

Introduction

Medulloblastoma (MB) is the most common malignant brain tumor in children. Despite recent improvements in overall survival, just a modest percentage of patients survive high-risk MB. The existing therapeutic approach to MB includes high doses of craniospinal radiation and chemotherapy. Though that approach has improved survival, 20%-30% of MB patients still succumb. Furthermore, patients with high-risk (such as c-Myc amplified) and metastatic tumors have an extremely poor survival rate. The devastating side-effects of radiation and chemotherapy substantially reduce qualify of life for surviving patients. Those facts underscore the urgency of developing new therapeutic regimens for treating MB. MicroRNAs (miRNAs) represent a possible target that can serve as an effective and less toxic therapeutic to treat medulloblastoma because they play key roles in chemo- and radiosensitivity. As described herein, an unbiased genomic screen was used to identify miR-584-5p as a potent tumor suppressor miRNA that inhibits MB stem cell growth and consequently growth and progression of MB in general and c-Myc-amplified MB in particular. MiR-584-5p sensitizes response to IR and VCR by damaging DNA and inducing spindle defects in MB cells. MiR-584-5p imparts its tumor-suppressing and therapy-sensitizing effects by targeting eIF4E3, HDAC1, and c-Myc in MB. These results show that if used as a therapeutic, miR-584-5p can inhibit high-risk MB growth and lower the VCR and radiation dose (and hence lower the associated toxic effects) required to kill MB cells.

Described herein are results using a high-throughput miRNA mimic screen in which miR-584-5p was identified as a potent tumor suppressor that makes vincristine (VCR), a microtubule-interfering chemotherapeutic agent routinely administered as a radiotherapy adjuvant to both high- and average-risk MB patients and ionizing radiation (IR) more effective in treating MB. Although miR-584-5p acts as a tumor suppressor in renal cell carcinoma, glioma, and neuroblastoma (Ueno, K. et al. Br J Cancer 104, 308-315, (2011); Wang, X. P., Deng, X. L. & Li, L. Y. Int J Clin Exp Pathol 7, 8573-8582 (2014); and Xiang, X. et al. Biochim Biophys Acta 1852, 1743-1754, (2015)), its role as a therapeutic adjuvant and underlying mechanism of action in cancer in general and MB in particular has not been investigated.

The results described herein show that miR-584-5p inhibited MB growth and prolonged survival of mice in an intracranial tumor xenograft model and miR-584-5p overexpression caused cell cycle arrest at G₂/M, DNA damage, and spindle defects leading to mitotic catastrophe in MB cells. miR-584-5p mediates its tumor suppressor and VCR/IR-sensitizing effect by targeting eukaryotic translation initiation factor 4e family member 3 (eIF4E3) and histone deacetylase 1 (HDAC1), thereby affecting cell cycle progression, microtubule dynamics, and DNA damage response. HDAC1 promotes MB growth (Canettieri, G. et al. Nat Cell Biol 12, 132-142, (2010)). Previous studies have shown that eIF4E3 is a translation initiation protein that may act as a tumor suppressor (Volpon, L., Osborne, M. J., Culjkovic-Kraljacic, B. & Borden, K. L. Cell Cycle 12, 1159-1160, (2013); and Osborne, M. J. et al. Proc Natl Acad Sci USA 110, 3877-3882, (2013)). Silencing either eIF4E3 or HDAC1 significantly inhibited MB growth and progression as well as improved sensitivity to IR and VCR. Furthermore, miR-584-5p overexpression or silencing of HDAC1/eIF4E3 inhibited MB stem cell proliferation and self-renewal without affecting normal neural stem cell growth. In MB tumor samples, reduced expression of miR-584-5p correlated with increased levels of HDAC1/eIF4E3. These results are the first to show a tumor-promoting and chemotherapy/IR-sensitizing function for eIF4E3 in MB. Furthermore, the results described herein are the first to show that a tumor suppressor miRNA can sensitize both VCR and IR responses by inducing spindle defects and mitotic catastrophe as well as DNA damage in MB. Taken together, the findings described herein identify a role for miR-584-5p/HDAC1/eIF4E3 in regulating DNA damage repair, microtubule dynamics, and stemness in MB. Furthermore, this study identifies new targets that can affect the therapeutic efficacy of IR and VCR and sets the stage for a new way to treat MB by using miR-584-5p therapeutically.

Compositions

Disclosed herein are compositions for the treatment of cancer or tumors. In an aspect, the cancer or tumors can express increased levels of histone deacetylase 1 (HDAC1) or eukaryotic translation initiation factor 4E family member 3 (eIF4E3). In an aspect, the composition can comprise a miR-584-5p and a microtubule-interfering chemotherapeutic agent. In an aspect, the composition can be a synergistic composition.

In an aspect, the cancer can be brain, myeloid, lymphoid, lung, breast, cancer, sarcoma or renal cancer. In some aspects, the cancer or tumor can express increased or higher levels of eIF4E3. In cancers or tumors that express increased or higher levels of eIF4E3, the cancer can be brain, medulloblastoma, myeloid, lymphoid, lung, breast, sarcoma or renal cancer. In some aspects, the cancer or tumor can express increased or higher levels of HDAC1. In cancers or tumors that express increased or higher levels of HDAC1, the cancer can be brain, medulloblastoma, myeloid, lymphoid, lung, breast, sarcoma or renal cancer. In an aspect, the cancer can be medulloblastoma.

MicroRNAs (miRNAs or MiRs) are a class of small (e.g., about 20 nucleotides in length), conserved non-coding RNAs that regulate mRNA degradation and translation, at least in part through binding to the 3′UTR of target genes. In an aspect, the target genes can be the HDAC1 gene and/or the EIF4E3 gene.

In an aspect, the miR-584-5p can be hsa-miR-584-5p. In an aspect, the miR-584-5p can comprise the nucleotide sequence UUAUGGUUUGCCUGGGACUGAG (SEQ ID NO: 1). In an aspect, the miR-584-5p can comprise the nucleotide sequence UUAUGGUUUGCCUGGGA (SEQ ID NO: 2). In an aspect, the composition can comprise a sequence derived from miR-584-5p. In an aspect, the miR-584-5p can consist of the nucleotide sequence UUAUGGUUUGCCUGGGACUGAG (SEQ ID NO: 1). In an aspect, the miR-584-5p can consist of the nucleotide sequence UUAUGGUUUGCCUGGGA (SEQ ID NO: 2). In an aspect, the composition can consist of a sequence derived from miR-584-5p. In an aspect, the composition can consist of a sequence derived from miR-584-5p, wherein the sequence derived from miR-584-5p has increased stability as compared to miR-584-5p.

In some aspects, the term “miR-584-5p” can also include fragments of the miR-584-5p molecule. As used herein, the term “fragment” refers to a portion of the full-length miR-584-5p. The size of the fragment can vary and must include a functional fragment, that is, the fragment must be able to modulate the expression of HDAC1 or EIF4E3 or components of the eIF4E3/HDAC1 signaling pathways and have therapeutic utility against HDAC1 or EIF4E3 expressing cancer cells as described herein. Typically, the fragment can comprise at least the seed region sequence AAACCATA (SEQ ID NO: 3). In some aspects, the fragment can comprise at least the seed region sequence AACCATAA (SEQ ID NO: 10)

In an aspect, the microtubule-interfering chemotherapeutic agent can be vincristine, paclitaxel or docetaxel.

Methods of Treatment

Disclosed herein, are methods of increasing sensitivity of one or more medulloblastoma cancer cells to radiotherapy or chemotherapy. The method can comprise: (a) identifying a subject in need of treatment; and (b) administering to the subject a therapeutically effective amount of a miR-584-5p. In an aspect, the therapeutically effective amount of a miR-584-5p can be administered before, during or after administration of radiotherapy or a chemotherapeutic agent. In an aspect, the therapeutically effective amount of a miR-584-5p can be in an amount sufficient to increase sensitivity of one or more medulloblastoma cancer cells to the radiotherapy or chemotherapeutic agent.

Disclosed herein, are methods of treating medulloblastoma in a subject, the method comprising: (a) identifying a subject in need of treatment; and (b) administering to the subject a therapeutically effective amount of a miR-584-5p. In an aspect, the methods can further comprise administering radiotherapy, a chemotherapeutic agent or both to the subject wherein the miR-584-5p increases the sensitivity of medulloblastoma cells to the radiotherapy, the chemotherapeutic agent or both in the subject.

Disclosed herein are methods of reducing growth of medulloblastoma cancer cells. The methods can comprise: (a) administering a therapeutically effective amount of a composition comprising miR-584-5p to a subject having or suspected of having cancer cells reduced in expression of miR-584-5p; and (b) administering to the subject a therapeutically effective amount of vincristine before, after or during administration of the composition comprising miR-584-5p, and wherein the medulloblastoma cancer cells have become sensitized to vincristine.

Disclosed herein are methods of diagnosing and treating cancer in a subject. Disclosed herein are methods comprising the steps in the following order: (a) obtaining a sample comprising tumor cells from a cancer patient; (b) measuring a level of miR-584-5p in one or more of the tumor cells of the sample; (c) identifying the cancer patient as a suitable candidate for treatment with a composition comprising miR-584-5p if the level of miR-584-5p is lower than a level of miR-584-5p in a control sample and identifying the cancer patient as an unsuitable candidate for treatment with a composition comprising miR-584-5p if the level of miR-584-5p is higher than a level of miR-584-5p in a control sample; and (d) administering the composition comprising miR-584-5p to the cancer patient identified as the suitable candidate, and not administering the composition comprising miR-584-5p to the cancer patient identified as the unsuitable candidate. In an aspect, the cancer can be medulloblastoma. In an aspect, the patient has medulloblastoma. In an aspect, the sample can be a biopsy. In an aspect, the method can further comprise administering radiotherapy, a chemotherapeutic agent or both to the cancer patient identified as the suitable candidate, wherein the composition comprising miR-584-5p increases the sensitivity of medulloblastoma cells to the radiotherapy, chemotherapeutic agent or both. In an aspect, the administration of the composition comprising miR-584-5p can render the cancer susceptible to a cytotoxic dose of the chemotherapeutic agent that can be lower than the cytotoxic dose required in the absence of the composition comprising miR-584-5p. In an aspect, the chemotherapeutic agent can be vincristine.

Disclosed herein are methods of inhibiting cancer or tumor growth. Disclosed herein are methods of inhibiting medulloblastoma growth. In an aspect, the methods can comprise: (a) identifying a subject in need of treatment; and (b) administering to the subject a therapeutically effective amount of a miR-584-5p before, during or after administration of radiotherapy and/or a chemotherapeutic agent, in an amount sufficient to inhibit cancer or tumor growth. In an aspect, the effective amount of a miR-584-5p administered before, during or after administration of radiotherapy and/or a chemotherapeutic agent can be in an amount sufficient to inhibit medulloblastoma growth.

Disclosed herein are methods of enhancing the efficacy of radiotherapy and/or a chemotherapeutic agent in a subject with medulloblastoma, the method comprising administering to the subject: (a) an effective amount of a chemotherapeutic agent and/or radiotherapy; and (b) a therapeutically effective amount of a miR-584-5p, wherein the administration of the miR-584-5p enhances the efficacy of the radiotherapy and/or the chemotherapeutic agent in the subject with medulloblastoma.

In some aspects, the methods include administering a therapeutically effective amount of a composition comprising miR-584-5p and administering to the subject a therapeutically effective amount of a chemotherapeutic agent before after or during administration of the composition comprising miR-584-5p. In some aspects, the methods can also include the administration of radiotherapy.

In some aspects, the methods described herein can further comprise administering a radiotherapy. In an aspect, the administration of a composition comprising miR-584-5p can increase the sensitivity of the medulloblastoma cancer cells to the radiotherapy.

In an aspect, the administration of a composition comprising miR-584-5p can increase the sensitivity of the medulloblastoma cancer cells to a chemotherapeutic agent.

In an aspect, the subject in need of treatment has been diagnosed with medulloblastoma prior to the administering step.

Medulloblastoma is a tumor in developing cells of the cerebellum that primarily affects children younger than 16, with 70% occurring in children under 10^{2,5}. The primary medulloblastoma tumor can occur in the cerebellum, a region of the brain at the base of the skull, and can spread to other parts of the brain and spinal cord. Medulloblastoma can be classified as high-risk in the following circumstances: the patient is younger than 3 years old; the tumor has spread to other parts of the brain; tumor cells have amplified c-Myc expression; and a large portion of the tumor (>1 cm3) remains after surgery.

The current standard of care for medulloblastoma involves the surgical removal (resection) of as much of the tumor as possible. Surgery can be followed with radiation therapy for all patients, although the dose can be higher for those patients whose tumor was not completely removed. Surgery can also be followed with chemotherapeutics, such as vincristine. Although this treatment strategy has prolonged survival for many patients, it can result in severe, permanent neurological side effects. The disclosed compositions and methods significantly improve the current standard of care as follows: increase the sensitivity of the tumor cells to radiation and chemotherapy treatments; allows for lower dosages of radiation and chemotherapy, thus reducing the deleterious side effects; and may result in a more complete eradication of the tumor cells, thus improving the long term recurrence and survival rates.

In an aspect, the medulloblastoma cancer can be wingless, Sonic Hedgehog, group 3 or group 4 subtype. In an aspect, the medulloblastoma can be pediatric medulloblastoma. In aspect, the medulloblastoma can be adult medulloblastoma.

In an aspect, the methods described herein can sensitize one or more medulloblastoma cancer cells to radiotherapy or chemotherapy or both by contacting one or more medulloblastoma cancer cells with an effective amount of miR-584-5p, a precursor or a sequence derived thereof.

In an aspect, the methods described herein can include the administration of miR-584-5p or variants thereof. Variants can include nucleotide sequences that are substantially similar to sequences of miR-584-5p, precursors or sequences derived thereof. In an aspect, variants include nucleotide sequences that are substantially similar to the miR-584-5p sequence or fragments thereof, including the miR-584-5p seed sequence. Variants can also include nucleotide sequences that are substantially similar to sequences of miRNA disclosed herein. A “variant” can mean a difference in some way from the reference sequence other than just a simple deletion of an N- and/or C-terminal nucleotide. Variants can also or alternatively include at least one substitution and/or at least one addition, there may also be at least one deletion. In some aspects, the variant miRNA to be administered can comprise a sequence displaying at least 80% sequence identity to the sequence of miR-584-5p (SEQ ID NO: 1 or 2). In some aspects, the miRNA to be administered can comprise a sequence displaying at least 90% sequence identity to SEQ ID NO: 1 or 2. In some aspects, the miRNA to be administered can comprise a sequence displaying at least 91%, 92%, 93%, 94%, 95%, 96%, 97%, 98% or 99% sequence identity to SEQ ID NO: 1 or 2. Alternatively or in addition, variants can comprise modifications, such as non-natural residues at one or more positions with respect to the miR-584-5p sequence. In an aspect, the variant can be a sequence wherein the last nucleotide of the miRNA is changed. In some aspects, the variant can be a sequence comprising at least one, at least two, or at least three substitutions at the 5′ end of the miR-584-5p. In an aspect, nucleotide substitutions can include nucleotide substitutions to the reference sequence which increase stability of the miR-584-5p or a variant thereof. In an aspect, nucleotide substitutions can be those which permit conjugation of the miR-584-5p or a variant thereof to a polymer or copolymer for forming a nanoparticle. Nucleotide substitutions can be substitutions of one or two bases. In some aspects, nucleotide substitutions can be substitutions of three bases. Deletions and insertions can include from one (1) to about three (3) bases.

Substitutions, deletions, insertions or any combination thereof may be used to arrive at a final derivative or variant. Generally, these changes are done on a few nucleotides to minimize the alteration of the molecule. However, larger changes may be tolerated in certain circumstances.

Generally, the nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, or 3 4 nucleotide base changes as compared to the parent or reference sequence of the invention, and shares or improves biological function, specificity and/or activity of the parent sequence. In some aspects, the parent or reference sequence can be miR-584-5p.

In some aspects, any of sequences disclosed herein can include a single nucleotide change as compared to the parent or reference sequence. In some aspects, any of the sequences disclosed herein can include at least two nucleotide changes as compared to the parent or reference sequence. The nucleotide identity between individual variant sequences can be at least 60%, 65%, 70%, 75%, 80%, 85%, 90%, 91%, 92%, 93%, 94%, 95%, 96%, 97%, 98%, 99% or 100%. Thus, a “variant sequence” can be one with the specified identity to the parent sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence.

As disclosed herein, miR-584-5p can sensitize cancers and cells to the administration of radiotherapy or a chemotherapeutic agent. Cancers and cells can be sensitized to a variety of radiotherapies or chemotherapeutic agents. Examples of radiotherapies can include one or more of craniospinal irradiation, three-dimensional conformal radiation therapy, intensity modulated radiation therapy (IMRT) and conformal proton beam radiation therapy. Examples of chemotherapeutic agents can include one or more of vincristine, vinblastine, paclitaxel and docetaxel. In an aspect, the chemotherapeutic agent can be vincristine. Other suitable radiotherapies and chemotherapeutic agents will be known to those skilled in the art and the scope of the instant disclosure is not limited by reference to the specific therapies or agents disclosed herein.

Generally, the sensitization can render the one or more cancer cells susceptible to a cytotoxic dose of the radiotherapy or a chemotherapeutic agent that can be lower than the cytotoxic dose required in the absence of the miR-584-5p. In an aspect, the administration of the miR-584-5p renders the one or more medulloblastoma cancer cells susceptible to a cytotoxic dose of the radiotherapy or chemotherapeutic agent. In an aspect, the administration of the miR-584-5p can render the one or more medulloblastoma cancer cells susceptible to a cytotoxic dose of the radiotherapy or chemotherapeutic agent that can be lower than the cytotoxic dose required in the absence of the miR-584-5p. In an aspect, the administration of the miR-584-5p can render the medulloblastoma cells susceptible to a cytotoxic dose of the radiotherapy or chemotherapeutic agent that can be lower than the cytotoxic dose required in the absence of the miR-584-5p. In an aspect, the therapeutically effective amount of vincristine can be lower than the therapeutically effective amount required in the absence of the composition comprising miR-584-5p. In an aspect, the administration of the chemotherapeutic agent can be before, during or after the administration of the miR-584-5p. In an aspect, the methods described herein can comprise administering both radiotherapy and a chemotherapeutic agent. In an aspect, the administration of a composition comprising miR-584-5p can increase the sensitivity of the medulloblastoma cancer cells to both the chemotherapeutic agent and radiotherapy. In an aspect, the methods described herein can comprise administering both radiotherapy and vincristine. In an aspect, the administration of a composition comprising miR-584-5p can increase the sensitivity of the medulloblastoma cancer cells to both vincristine and radiotherapy.

In some aspects, the methods disclosed herein can result in a reduction in one or more the side effects of the chemotherapeutic agent and/or the radiotherapy or a reduction in the severity of one or more of the side effects of the chemotherapeutic agent and/or the radiotherapy. Examples of side effects associated with 35.5 Gy followed by up to 50-Gy radiation boosts include but are not limited to neurocognitive deficits, chronic neuropathy, chronic hypopituitarism and secondary malignancies. Examples of side effects associated with the administration of side effects of vincristine include but are not limited to neurotoxicity that includes, but is not limited to, sensorimotor and autonomic neuropathy, hearing loss, mononeuropathy, and seizures.

In some aspects, the methods disclosed herein can reduce the dose of the radiation or chemotherapeutic agent or both by 10-fold, 11-fold, 12-fold, 13-fold, 14-fold, 15-fold, 16-fold, 17-fold, 18-fold, 19-fold, 20-fold, etc., or any amount in between. In an aspect, the dose of the radiotherapy can be lower than the current dose schedule. In an aspect, the dose of the vincristine can be lower than the current dose schedule.

The cancers and cancer cells to which aspects disclosed herein relate can be resistant or sensitive or naïve (cancers or cells that have not been previously exposed to a particular radiotherapy or chemotherapeutic agent).

In some aspects, the cancer can have reduced expression of miR-584-5p when compared to a reference sample before the administration of a composition comprising miR-584-5p. In some aspects, the medulloblastoma cancer cells have reduced expression of miR-584-5p when compared to a reference sample before the administration of the composition comprising miR-584-5p. In some aspects, the methods described herein can further comprise determining the level of miR-584-5p in the cancer or cancer cells before the administration of a composition comprising miR-584-5p wherein the level of miR-584-5p can be lower when compared to a reference sample. In some aspects, the methods described herein can further comprise determining the level of miR-584-5p in the medulloblastoma cancer cells before the administration of a composition comprising miR-584-5p wherein the level of miR-584-5p can be lower when compared to a reference sample.

In some aspects, the cancer cell or cells can be obtained from a sample (e.g., a biopsy or blood sample from the subject) and the level of expression of miR-584-5p in the cell or cells of interest (from the sample) can be compared to a reference sample or a control cell or cells. The reference sample or control cell(s) can be non-cancerous and can be generally of the same type as the cell or sample. In some aspects, the reference sample can be a normal (non-cancerous) cell from the subject from whom the tissue or a cell that is suspected of being cancerous is obtained. The reference sample can be, but is not required to be from the same subject. In some aspects, the reference sample can be provided from a different subject who is established or known to not have the specific cancer being compared. In some aspects, the reference sample can be sex-matched, age-matched and/or race-matched to the subject whose sample is being compared. In some aspects, the reference sample can be the mean expression level (as a measure of the number of cells used) obtained from the expression levels of miR-584-5p from a number of individuals, wherein the same histological type as the cell being assayed is used to measure the miR-584-5p expression level, and wherein all of the individuals used to obtain the mean expression or miR-584-5p expression level do not have cancer. In this case, the individuals can be from different age groups, races and sexes. Alternatively, the individuals may be from the same age groups, race and/or sex.

As used herein, the term “expression,” when used in the context of determining or detecting the expression or expression level of one or more genes, can refer to determining or detecting transcription of the gene (i.e., determining mRNA levels) and/or determining or detecting translation of the gene (e.g., determining or detecting the protein produced). To determine the expression level of a gene means to determine whether or not a gene is expressed, and if expressed, to what relative degree. The expression level of one or more genes disclosed herein can be determined directly (e g, immunoassays, mass spectrometry) or indirectly (e.g., determining the mRNA expression of a protein or peptide). Examples of mass spectrometry include ionization sources such as EI, CI, MALDI, ESI, and analysis such as Quad, ion trap, TOF, FT or combinations thereof, spectrometry, isotope ratio mass spectrometry (IRMS), thermal ionization mass spectrometry (TIMS), spark source mass spectrometry, Multiple Reaction Monitoring (MRM) or SRM. Any of these techniques can be carried out in combination with prefractionation or enrichment methods. Examples of immunoassays include immunoblots, Western blots, Enzyme linked Immunosorbant Assay (ELISA), Enzyme immunoassay (EIA), radioimmune assay Immunoassay methods use antibodies for detection and determination of levels of an antigen are known in the art. The antibody can be immobilized on a solid support such as a stick, plate, bead, microbead or array.

Expression levels of one or more of the genes described herein can be also be determined indirectly by determining the mRNA expression for the one or more genes in a tissue sample. RNA expression methods include but are not limited to extraction of cellular mRNA and Northern blotting using labeled probes that hybridize to transcripts encoding all or part of the gene, amplification of mRNA using gene-specific primers, polymerase chain reaction (PCR), and reverse transcriptase-polymerase chain reaction (RT-PCR), followed by quantitative detection of the gene product by a variety of methods; extraction of RNA from cells, followed by labeling, and then used to probe cDNA or olignonucleotides encoding the gene, in situ hybridization; RNA-sequencing; and detection of a reporter gene.

Methods to measure protein expression levels include but are not limited to Western blot, immunoblot, ELISA, radioimmunoassay, immunoprecipitation, surface plasmon resonance, chemiluminescence, fluorescent polarization, phosphorescence, immunohistochemical analysis, microcytometry, microarray, microscopy, fluorescence activated cell sorting (FACS), and flow cytometry. The method can also include specific protein property-based assays based including but not limited to enzymatic activity or interaction with other protein partners. Binding assays can also be used, and are well known in the art. For instance, a BTAcore machine can be used to determine the binding constant of a complex between two proteins. Other suitable assays for determining or detecting the binding of one protein to another include, immunoassays, such as ELISA and radioimmunoassays. Determining binding by monitoring the change in the spectroscopic can be used or optical properties of the proteins can be determined via fluorescence, UV absorption, circular dichroism, or nuclear magnetic resonance (NMR). Alternatively, immunoassays using specific antibody can be used to detect the expression on of a particular protein on a tumor cell.

As used herein, the term “reference,” “reference expression,” “reference sample,” “reference value,” “control,” “control sample” and the like, when used in the context of a sample or expression level of one or more genes or proteins or microRNAs refers to a reference standard wherein the reference is expressed at a constant level among different (i.e., not the same tissue, but multiple tissues) tissues, and is unaffected by the experimental conditions, and is indicative of the level in a sample of a predetermined disease status (e.g., not suffering from cancer). The reference value can be a predetermined standard value or a range of predetermined standard values, representing no illness, or a predetermined type or severity of illness.

Reference expression can be the level of the one or more genes or proteins or microRNAs described herein in a reference sample from a subject, or a pool of subjects, not suffering from cancer or from a predetermined severity or type of cancer. In an aspect, the reference value can be the level of one or more genes or proteins or microRNAs disclosed herein in the tissue of a subject, or subjects, wherein the subject or subjects is not suffering from cancer.

Determining the expression level of one or more genes or proteins or microRNAs disclosed herein can include determining whether the gene or proteins or microRNAs is upregulated or increased as compared to a control or reference sample, downregulated or decreased (e.g., low) compared to a control or reference sample, or unchanged compared to a control or reference sample. As used herein, the terms, “upregulated” and “increased expression level” or “increased level of expression” refers to a sequence corresponding to one or more genes or proteins or microRNAs disclosed herein that is expressed wherein the measure of the quantity of the sequence exhibits an increased level of expression (e.g., high) when compared to a reference sample or “normal” control. For example, the terms, “upregulated” and “increased expression level” or “increased level of expression” refers to a sequence corresponding to one or more genes disclosed herein that is expressed wherein the measure of the quantity of the sequence exhibits an increased level of expression of one or more of protein(s) and/or mRNA when compared to the expression of the same mRNA(s) from a reference sample or “normal” control. An “increased expression level” refers to an increase in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more. As used herein, the terms “downregulated,” “decreased level of expression,” or “decreased expression level” refers to a sequence corresponding to one or more genes or proteins or microRNAs disclosed herein that is expressed wherein the measure of the quantity of the sequence exhibits a decreased level of expression when compared to a reference sample or “normal” control. For example, the terms “downregulated,” “decreased level of expression,” or “decreased expression level” refers to a sequence corresponding to one or more genes disclosed herein that is expressed wherein the measure of the quantity of the sequence exhibits a decreased level of expression of one or more protein(s) and/or mRNA when compared to the expression of the same mRNA(s) from a reference sample or “normal” control. A “decreased level of expression” refers to a decrease in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more.

In some aspects, a relative level of EIF4E3 or HDAC1 can be measured. High or increased levels of EIF4E3 or HDAC1 correspond to low levels miR-584-5p.

In some aspects, the efficacy of the anti-cancer treatments can be determined. The levels of miR-584-5p can serve as a biomarker for the efficacy of the anti-cancer treatment. For example, the levels of miR-584-5p in the post-treatment cells can be higher than the levels of miR-584-5p in the pre-treatment cells, then the treatment can be deemed to be effective.

In an aspect, the miR-584-5p can be hsa-miR-584-5p. In an aspect, the miR-584-5p can comprise the nucleotide sequence UUAUGGUUUGCCUGGGACUGAG (SEQ ID NO: 1). In an aspect, the miR-584-5p can comprise the nucleotide sequence UUAUGGUUUGCCUGGGA (SEQ ID NO: 2). In an aspect, the composition can comprise a sequence derived from miR-584-5p.

Disclosed herein, are methods of suppressing expression of a target gene in a cell. In an aspect, the methods can comprise contacting a cell with miR-584-5p, or a variant thereof. In an aspect, the target gene encodes HDAC1 or eIF4E3; thereby suppressing the expression of the target gene in cell when compared to a reference sample. In an aspect, the cell can be a cancer cell. In aspect, the cell can be a medulloblastoma cell. The method can include contacting a cell with a therapeutically effective amount of miR-584-5p.

Contacting a cell or cancer or cancer cell with a miR-584-5p, a variant thereof or molecule capable of stimulating or enhancing the expression or activity of a miR-584-5p can be achieved by any method known in the art. In some aspects, contacting the cell and the miR-584-5p occur in vivo. The miR-584-5p or molecule capable of stimulating or enhancing the expression or activity of a miR-584-5p or molecule may be contacted with the cell directly, for example, applied directly to a cell requiring sensitizing to a chemotherapeutic agent or radiotherapy, or alternatively can be combined with the cell indirectly, e.g. by injecting the molecule into the bloodstream of a subject, which then carries the molecule to the cell requiring sensitizing to a chemotherapeutic agent or radiotherapy. Further, a sample can be removed from a subject and combined with miR-584-5p or molecule capable of stimulating or enhancing the expression or activity of a miR-584-5p in vitro prior to returning at least a portion of the sample back to the subject. For example, the sample can be a blood sample which can be removed from a subject and combined with the miR-584-5p prior to injecting at least a portion of the blood back into the subject.

The compositions described herein can be formulated to include a therapeutically effective amount of miR-584-5p, or a variant thereof described herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of cancer.

The compositions described herein can be formulation in a variety of combinations. The particular combination of miR-584-5p, or a variant thereof with radiotherapy and one or more of chemotherapeutic agents can vary according to many factors, for example, the particular the type and severity of the cancer.

The compositions described herein can be administered to the subject (e.g., a human patient) in an amount sufficient to delay, reduce, or preferably prevent the onset of clinical disease. Accordingly, in some aspects, the patient can be a human patient. In an aspect, the human subject or patient can be a child or an adult. In therapeutic applications, compositions are administered to a subject (e.g., a human patient) already with or diagnosed with cancer in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the cancer is delayed, hindered, or prevented, or the cancer or a symptom of the cancer is ameliorated. One or more of the symptoms can be less severe. Recovery can be accelerated in an individual who has been treated.

In some aspects, the cancer can be a primary or secondary tumor. In an aspect, the cancer can be a metastatic tumor. In other aspects, the primary or secondary tumor can within the patient's brain, breast, blood, lung, kidney, lymphatic system, connective tissue. In yet other aspects, the cancer has metastasized.

Disclosed herein, are methods of treating a patient with cancer. The cancer can be any cancer. In some aspects, the cancer can breast cancer, lung cancer, brain cancer, liver cancer, blood cancer, lymphatic cancer or kidney cancer. In an aspect, the subject has been diagnosed with cancer prior to the administering step. In an aspect, the cancer can be brain cancer. In an aspect, the cancer can be medulloblatoma.

The compositions described herein can be formulated to include a therapeutically effective amount of miR-584-5p, or a variant thereof alone or in combination with one or more of the chemotherapeutic agents disclosed herein. In an aspect, miR-584-5p, or a variant thereof can be contained within a pharmaceutical formulation. In an aspect, the pharmaceutical formulation can be a unit dosage formulation.

The therapeutically effective amount or dosage of the miR-584-5p, or a variant thereof any of the radiotherapies, and chemotherapeutic agents used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the cancer symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

Dosages of vincristine during radiation can be in the range of 1.5 to 2 mg/m² or any amount in between, once a week for about 6 weeks. Dosages of vincristine after radiation can be in the range of 1.5 to 2 mg/m² or any amount in between, once a week for about 3 weeks. Dosages of radiation can be about 35 Gy to the central nervous system and about 50 Gy to boost the posterior fossa.

Compositions comprising miR-584-5p can be administered to a subject in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1500, 2000, 2500, 3000 μg or mg, or any range between 0.5 μg or mg and 3000 μg or mg. The amount specified can be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area). A clinician can readily determine the effective amount of a miR-584-5p—i.e. the amount of miR-584-5p, or a variant thereof needed to inhibit proliferation of a cancer cell, by taking into account factors, such as the size and weight of the subject; the extent of disease penetration; the age, health and sex of the subject; the route of administration; and whether the administration is regional or systemic.

In an aspect, the dosages of miR-584-5p, radiotherapy or any of the chemotherapeutic agents disclosed herein can be less when combined with one or more of the compounds disclosed herein.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The compositions described herein can be administered in conjunction with other therapeutic modalities to a subject in need of therapy. The miR-584-5p, or a variant thereof can be given prior to, simultaneously with or after treatment with other agents or regimes. In an aspect, miR-584-5p, or a variant thereof can be given prior to, simultaneously or during, or after administration of one or more radiotherapies or one or more chemotherapeutic agents or a combination of both. For example, miR-584-5p, or a variant thereof alone or with any of the treatments or agents disclosed herein can be administered in conjunction with standard therapies used to treat cancer. In an aspect, miR-584-5p, or a variant thereof can be administered or used together with one or more chemotherapy agents, radiotherapy or a combination thereof.

In an aspect, miR-584-5p, or a variant thereof and one or more chemotherapeutic agents can be co-formulated. The compositions described herein can be formulated to include a therapeutically effective amount of miR-584-5p in combination with one or more of the chemotherapeutic agents disclosed herein. In an aspect, the chemotherapeutic agents can be vincristine, paclitaxel, or docetaxel. In an aspect, miR-584-5p, or a variant thereof and one or more chemotherapeutic agents disclosed herein can be co-formulated inside a nanoparticle. In an aspect, miR-584-5p, or a variant thereof can be contained within a pharmaceutical formulation. In an aspect, the pharmaceutical formulation can be a unit dosage formulation.

In an aspect, the methods of treatment disclosed herein can also include the administration of a therapeutically effective amount of immunotherapy, stem cell transplantation or a combination thereof. The combination therapies disclosed can be administered as one or more pharmaceutical compositions and, if separately, can be administered simultaneously or sequentially in any order.

In an aspect, any of the compositions disclosed herein can be administered with one or more immunotherapeutic or immune modulating agents. As used herein, the terms “immunomodulator” and “immune modulating agents” refer to a component (e.g., a protein, peptide, pharmacological and/or immunological agent) that modifies (e.g., potentiates) the immune system response toward a desired immune system response. An immunomodulator can also be an adjuvant. The immunomodulator can be a therapeutic agent that specifically or nonspecifically augments an immune system response. Examples of immunomodulators or immune modulating agents include but are not limited to cytokines, interleukins, chemokines or any protein, peptide, pharmacological or immunological agent that provides an increase in an immune system response. Examples of immunotherapeutic agents can include but are not limited antibody therapy, cytokine therapy, and combination immunotherapy. In an aspect, the immune modulating agent can be a type 1 interferon such as interferon alpha or interferon beta. Other immune modulating agents include but are not limited to anti-CD40 ligand antibody, Flt3 ligand, CD200, TGFβ, PDL1, PDL2, soluble CD83, OX40L, anti-IL-17 antibody, IL-2, IL-10, IL-12, IL-19, IL-33, galectin-1, CTLA-4, CD103 and indoleamine 2,3-dioxygenase. The compositions described herein can be a combination therapy for a disease.

The miR-584-5p, or a variant thereof can be administered as “combination” therapy. It is to be understood that, for example, miR-584-5p, or a variant thereof can be provided to the subject in need, either prior to administration of a radiation therapy or chemotherapeutic agent or any combination thereof, concomitant with administration of said radiation therapy, and/or chemotherapeutic agent or any combination thereof (co-administration) or shortly thereafter.

In an aspect, cancer cells can be sensitized prior to the administration of a radiation therapy and/or a chemotherapeutic agent or any combination thereof comprising administering to a subject in need an amount (e.g., a therapeutic amount) of a radiation therapy and/or a chemotherapeutic agent or any combination thereof in combination with an amount (e.g., a sensitizing amount; or an amount that is less than what is typically recommended) of miR-584-5p.

Pharmaceutical Compositions

As disclosed herein, are pharmaceutical compositions, comprising miR-584-5p and a pharmaceutical acceptable carrier described herein. In some aspects, miR-584-5p can be formulated for oral or parental administration. In an aspect, the parental administration is intravenous, subcutaneous, intramuscular or direct injection. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes miR-584-5p dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

In an aspect, miR-584-5p can be administered systemically. In an aspect, miR-584-5p can be administered intravenously. In an aspect, the pharmaceutical composition can be formulated for systemic or intravenous administration. In an aspect, the composition can be formulated in a lipid emulsion. In an aspect, the miR-584-5p can be formulated for delivery in a lipid emulsion, a liposome, a nanoparticle, an exosome, or in a viral vector. The liposome can be a unilamellar, multilamellar, or multivesicular liposome. A wide variety of liposomes and exosomes can be used. For example, in some aspects, a silicone nanoparticle can be used to deliver a miR-584-5p to a cell. In some aspects, a nanovector can be used to deliver a miR-584-5p to a subject. In some aspects, compositions comprising miR-584-5p can be administered into a tumor locus by a stereotaxic apparatus.

In an aspect, miR-584-5p can be encoded by a nucleic acid. The nucleic acid can be transfected into one or more cells. The transfection can comprise electroporation or incubation with a viral vector. In an aspect, the nucleic acid can be located in a vector. In an aspect, the vector can be plasmid, cosmid, phagemid or a viral vector. In an aspect, the vector can comprise a lipid, lipid emulsion, liposome, nanoparticle or exosomes. In an aspect, nucleic acid can be comprised in a lipid, lipid emulsion, liposome, nanoparticle or exosome. In an aspect, viral vector can be an adenovirus, an adeno-associated virus, a lentivirus or a herpes virus. In an aspect, the vector can comprise a lipid, lipid emulsion, liposome, nanoparticle or exosomes. Nanoparticles. The compositions described herein can comprise one or more nanoparticles. The nanoparticle compositions disclosed herein can be used to enhance delivery of conjugated or entrapped miR-584-5p across the blood brain barrier. Examples of nanoparticles (used interchangeably with the term “nanocarrier”) can be found, for example, in U.S. Patent Publication No. 2010-0233251. Examples of nanocarriers include, but are not limited to nanocarriers comprising one or more polymers. In some aspects, the one or more polymers can be a water soluble, non-adhesive polymer. In some aspects, the polymer can be polyethylene glycol (PEG) or polyethylene oxide (PEO). In some aspects, the polymer can be a polyalkylene glycol or polyalkylene oxide. In some aspects, the one or more polymers can be a biodegradable polymer. In some aspects, the one or more polymers can be a biocompatible polymer that can be a conjugate of a water soluble, non-adhesive polymer and a biodegradable polymer. In some aspects, the biodegradable polymer can be polylactic acid (PLA), poly(glycolic acid) (PGA), or poly(lactic acid/glycolic acid) (PLGA). In some aspects, the nanocarrier can be composed of PEG-PLGA polymers.

In some aspects, the nanocarrier can be formed by self-assembly. Self-assembly refers to the process of the formation of a nanocarrier using components that will orient themselves in a predictable manner forming nanocarriers predictably and reproducibly. In some aspects, the nanocarriers can be formed using amphiphillic biomaterials which orient themselves with respect to one another to form nanocarriers of predictable dimension, constituents, and placement of constituents. In some aspects, the nanocarrier can be a microparticle, nanoparticle, or picoparticle. In some aspects, the microparticle, nanoparticle, or picoparticle can be self-assembled.

In some aspects, the nanocarrier can have a positive zeta potential. In some aspects, the nanocarrier can have a net positive charge at neutral pH. In some aspects, the nanocarrier can comprise one or more amine moieties at its surface. In some aspects, the amine moiety can be a primary, secondary, tertiary, or quaternary amine. In some aspects, the amine moiety can be an aliphatic amine. In some aspects, the nanocarrier can comprise an amine-containing polymer. In some aspects, the nanocarrier can comprise an amine-containing lipid. In some aspects, the nanocarrier can comprise a protein or a peptide that can be positively charged at neutral pH. In some aspects, the nanocarrier can be a latex particle. In some aspects, the nanocarrier with the one or more amine moieties on its surface can have a net positive charge at neutral pH.

Nanoparticles can aid the delivery of the miR-584-5p. Delivery can be to a particular site of interest, e.g., the medulloblastoma cancer cells. In some aspects, the nanoparticle can create a timed release of the miR-584-5p to enhance and/or extend the therapeutic response. In some aspects, the nanoparticle can be associated with the miR-584-5p such that the composition can elicit increasing the sensitivity of one or more medulloblastoma cancer cells to radiotherapy or chemotherapy. The association can be, for example, wherein the nanoparticle can be coupled or conjugated with the miR-584-5p. The terms “coupled” and “conjugated” are meant that there is a chemical linkage between the nanoparticle and the miR-584-5p. In some aspects, the miR-584-5p can be entrapped or encapsulated within the nanoparticle. In some aspects, the miR-584-5p can be entrapped within the nanoparticle by a water/oil/water emulsion method. In some aspects, the nanoparticle can be poly(lactide co-glycolide) (PLGA). Depending on the ratio of lactide to glycolide used for the polymerization, different forms of PLGA can be obtained and utilized. These forms are typically identified in regard to the monomers' ratio used (e.g., PLGA 75:25 identifies a copolymer whose composition can be 75% lactic acid and 25% glycolic acid). Different ratios can be used in this invention, e.g., 90:10, 80:20, 70:30, 60:40, 50:50, 40:60, 30:70, 20:80, 10:90, and numbers above and in between these ratios. Additional examples of suitable nanoparticles include chitosin, calcium phosphate, lipids of various bacteria like E. coli, mycobactera, leptospira and mixtures thereof. In one example, the composition can be derived mixing about 180 mg of PLGA to about 5 mg of miR-584-5p (or about 36 mg PLGA to 1 mg miR-584-5p). The entrapment (encapsulation) efficiency of miR-584-5p can vary. In an aspect, the nanoparticle can be 50-55% entrapped/encapsulated, calculated based on amount of total miR-584-5p used in the entrapment. Entrapped miR-584-5p can be administered as mixtures of entrapped/encapsulated and unentrapped/unencapsulated miR-584-5p or the entrapped/encapsulated miR-584-5p can be further purified.

In some aspects, one or more ligands or antibodies can be surface-anchored to the nanoparticle. For example, the ligand or antibody can be selected based on the proteins expressed in medullobastoma compared to other normal or non-cancerous tissue (e.g., cerebellum). In an aspect, the ligand or antibody can have an affinity for CD24 that is highly expressed in medullobastoma compared to normal cerebellum. In some aspects, the ligand can be glyco-heptapeptide. In some aspects, the ligand or antibody can be modified to enhance cellular uptake across the blood brain barrier. Such modifications are within the ability of one of ordinary skill in the art. The presence of a selective ligand or antibody can help direct the composition to medulloblastoma cancer cells.

In some aspects, miR-584-5p can be conjugated to copolymer. Traditional copolymers have been used in numerous laboratories worldwide and also in several clinical trials. (See U.S. Pat. No. 5,037,883, which is hereby incorporated by reference in its entirety). For example, N-(2-hydroxypropyl)methacrylamide) (HPMA) copolymers are: (1) biocompatible and have a well-established safety profile; (2) water-soluble and have favorable pharmacokinetics when compared to low molecular weight (free, non-attached) drugs; and (3) possess excellent chemistry flexibility (i.e., monomers containing different side chains can be easily synthesized and incorporated into their structure). However, HPMA polymers are not degradable and the molecular weight of HPMA polymers should be kept below the renal threshold to sustain biocompatibility. This limits the intravascular half-life and accumulation of HPMA polymers in solid tumor via the EPR (enhanced permeability and retention) effect.

A backbone degradable HPMA copolymer carrier can be used to overcome limitations associated with HPMA. The copolymer carrier can contain enzymatically degradable sequences (i.e., by Cathepsin B, matrix matalloproteinases, etc.) in the main chain (i.e., the polymer backbone) and enzymatically degradable side chains (i.e., for drug release). (See, e.g., U.S. patent application Ser. No. 13/583,270, which is hereby incorporated by reference in its entirety). Upon reaching the lysosomal compartment of cells, the drug can be released and concomitantly the polymer carrier can be degraded into molecules that are below the renal threshold and can be eliminated from the subject. Thus, diblock or multiblock biodegradable copolymers with increased molecular weight can be produced. This can further enhance the blood circulation time of the copolymer-miR-584-5p therapeutic conjugate disclosed herein, which is favorable for drug-free macromolecular therapeutics targeting, for example, circulating cancer cells. Furthermore, U.S. Pat. No. 4,062,831 describes a range of water-soluble polymers and U.S. Pat. No. 5,037,883 describes a variety of peptide sequences, both of which are hereby incorporated by reference in their entireties.

In some instances, the miR-584-5p can be conjugated to HPMA copolymers administered in the disclosed methods can comprise 2, 3, 4, 5, 6, 7, 8, 9, or 10 HPMA copolymers. In some instances, each HPMA copolymer can be connected via enzymatically degradable peptides.

In some aspects, the miR-584-5p can be conjugated to HPMA copolymers administered in the disclosed methods can also comprise a linker. In some aspects, the linker can be a peptide linker.

Vectors can include plasmids, cosmids, and viruses (e.g., bacteriophage, animal viruses, and plant viruses), and artificial chromosomes (e.g., YACs). Vectors can comprise targeting molecules. A targeting molecule is one that directs the desired nucleic acid to a particular organ, tissue, cell, or other location in a subject's body. A vector, generally, brings about replication when it is associated with the proper control elements (e.g., a promoter, a stop codon, and a polyadenylation signal). Examples of vectors that are routinely used in the art include plasmids and viruses. The term “vector” includes expression vectors and refers to a vector containing a nucleic acid sequence coding for at least part of a gene product capable of being transcribed. A variety of ways can be used to introduce an expression vector into cells. In an aspect, the expression vector comprises a virus or an engineered vector derived from a viral genome. As used herein, “expression vector” is a vector that includes a regulatory region. A variety of host/expression vector combinations can be used to express the nucleic acid sequences disclosed herein. Examples of expression vectors include but are not limited to plasmids and viral vectors derived from, for example, bacteriophages, retroviruses (e.g., lentiviruses), and other viruses (e.g., adenoviruses, poxviruses, herpesviruses and adeno-associated viruses). Vectors and expression systems are commercially available and known to one skilled in the art.

Articles of Manufacture

The composition described herein can be packaged in a suitable container labeled, for example, for use as a therapy to treat cancer or any of the methods disclosed herein. Accordingly, packaged products (e.g., sterile containers containing the composition described herein and packaged for storage, shipment, or sale at concentrated or ready-to-use concentrations) and kits, including at least miR-584-5p as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compounds can be ready for administration (e.g., present in dose-appropriate units), and may include a pharmaceutically acceptable adjuvant, carrier or other diluent. Alternatively, the compounds can be provided in a concentrated form with a diluent and instructions for dilution.

In an aspect, the kits can include one or more of miR-584-5p or molecules derived from miR-584-5p; expression vectors comprising nucleic acid sequences encoding miR-584-5p or one or more molecules derived from miR-584-5p; reagents for preparing samples from blood samples or biopsy samples. The kit can include one or more pharmaceutically acceptable carriers. In addition, devices or materials for administration of the miR-584-5p (e.g., syringes (pre-filled with miR-584-5p), needles, liposomes, etc.) can also be included.

### EXAMPLES

### Example 1: High-Throughput Functional Screen Identifies miR-584-5p as a New Therapeutic Adjuvant for Improving the Efficacy of VCR in Treating MB

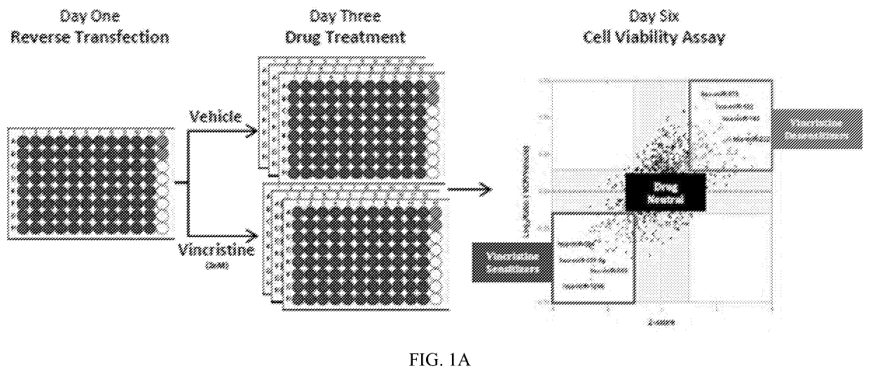

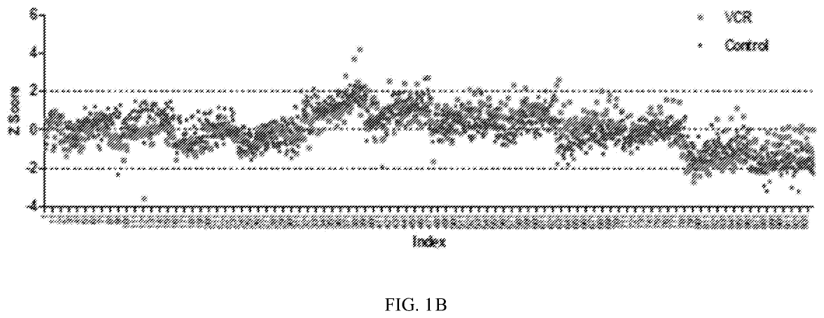

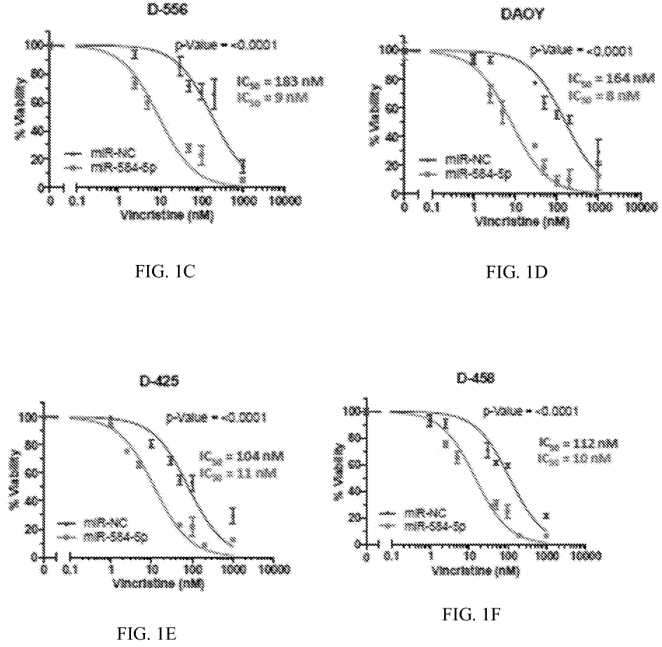

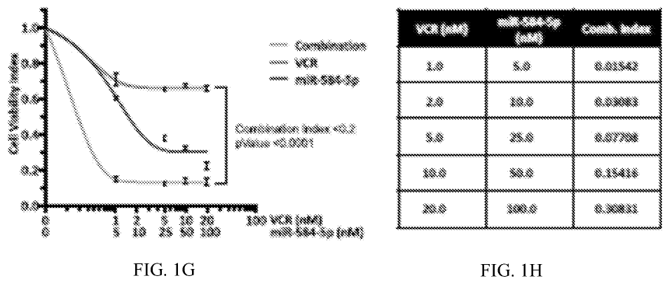

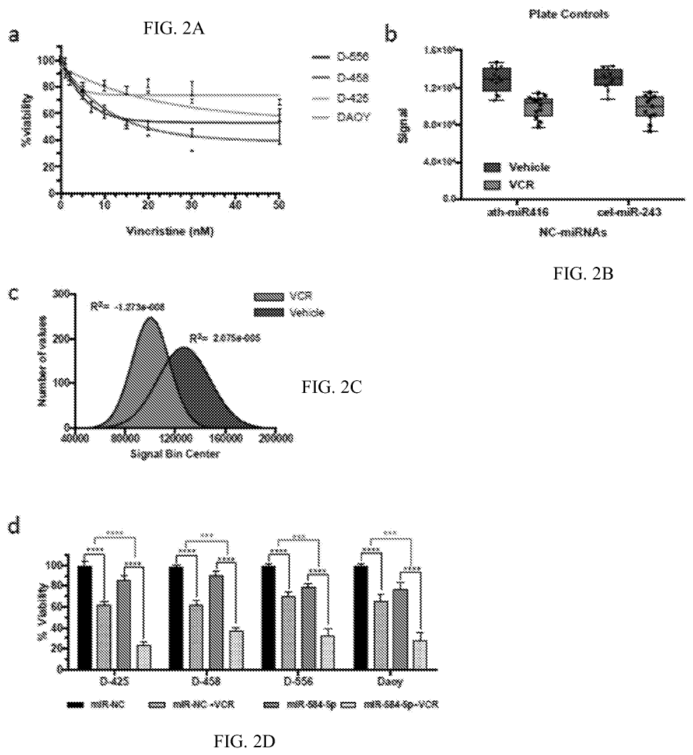

To identify miRNAs that may sensitize VCR response in MB, a high-throughput screening platform was combined with a library of 1,902 chemically synthesized human miRNA mimics. The miRNAs are arrayed in a one-miRNA-one-well format in 96-well microtiter plates. Reverse transfection of Group 3/c-Myc-amplified D458Med cells was performed in triplicate in the presence and absence of a sublethal dose of VCR, which was optimized in four MB cell lines before the screen (FIG. 2A). Cells were subjected to VCR at an IC20 lethal concentration for 72 hours after 48 hours of transfection, and cell viability was measured (FIG. 1A). Candidate miRNAs were prioritized for validation by functional and interaction assays using standard Student t-tests (with pooled variance), false discovery rate (Q=0.5%), and by using the magnitude of response (lowest 2.5 percentile of the distribution of the μ_{VCR}/μ_{Control} ratios).

The screen yielded three categories of miRNAs:
1. Sensitizers, which decreased the MB cell viability in the presence of VCR in comparison with carrier;
2. Desensitizers, which increased MB cell viability in the presence of VCR compared in comparison with carrier; and
3. Drug neutral, which either significantly (>25%) increased or decreased cell viability in carrier itself and therefore did not affect VCR therapy (FIGS. 1A, B and FIGS. 2B, C).

Screen results were also validated using additional MB cell lines: D425Med, D556Med, and DAOY (FIG. 2D, and data not shown). The drug sensitizer miR-584-5p, showed the highest magnitude of difference in cell viability in VCR-treated MB cells in comparison with untreated cells. To further confirm the VCR-sensitizing effect of miR-584-5p, MB cells (D425Med, D556Med, D458Med, and DAOY) were treated with miR-584-5p mimic in the absence and presence of increasing concentrations of VCR. Combining miR-584-5p and VCR resulted in ˜10- to 20-fold lower 50% inhibitory concentration than that in control in all four cell lines (FIGS. 1C-F). MB cells were transfected with miR-NC or miR-584 mimic followed by treatment with VCR or vehicle for 72 hours. Cell viability was assessed using alamarBlue cell viability assay. The p-values were determined by the sum-of-squares F test. Error bars represent mean±standard error of the mean (SEM). Consistent with that finding, combination treatment with increasing concentration of miR-584-5p mimic and increasing concentrations of VCR showed a highly synergistic effects on MB cells for all treatment combinations (www.combosyn.com) (Chou, T. C. Cancer Res 70, 440-446, (2010)) (FIGS. 1G, H). Compusyn software (http://www.combosyn.com/) was used to calculate combination indices (CIs). The p-values were determined by the sum-of-squares F test. Error bars represent mean±SEM.

Cell lines and cell culture. D556Med, D425Med, and D485Med cell lines were used. DAOY was obtained from the American Type Culture Collection (Manassas, Va., USA). Cells were cultured at 37° C. in a 5% CO₂ humidified atmosphere in minimal essential medium plus 10% fetal calf serum, 1% nonessential amino acids, 1% 1-glutamine, 100 IU of penicillin per ml, and 100 μg of streptomycin per ml. Human iPSC-derived neural stem cells and neurons were also used.

miRNA mimic library screen. The primary screen was conducted using MI00200 MISSION microRNA Mimic v.17, in which miRNA mimics were arrayed in a one-miRNA-one-well format in 96-well microtiter plates. Reverse transfection of 10³ D458Med cells was performed in triplicate. Forty-eight hours after transfection, cells were treated for 72 hours with a 20% inhibitory concentration (2 nM) of VCR. Cell viability was assessed using CellTiter-Glo (Promega, Madison, Wis., USA) according to the manufacturer's protocol. ath-miR-416 or cel-miR-243 was added as a negative control, whereas siRNA against PLK-1 was used as a positive control in each plate. Candidate miRNAs were prioritized for validation by functional and interaction assays using t-tests (with pooled variance), false discovery rate (Q=0.5%), and by using the magnitude of response (lowest 2.5 percentile of the distribution of the μ_{VCR}/μ_{Control} ratios).

miRNA, siRNA, and plasmid transfection. miR-584-5p and miR-NC were purchased from Thermofisher; si-EIF4E3, si-HDAC1, si-MYC, and scrambled-siRNA were purchased from Sigma. MB cell lines were transfected using Lipofectamine RNAiMAX Reagent (Thermofisher) according to the manufacturer's protocol. eIF4E3 plasmid was purchased from Origene (#RC209694). HDAC1 (#13820) and c-Myc (#46970) plasmids were purchased from Addgene. MB cell lines were transfected using Lipofectamine 2000 (Thermofisher) according to the manufacturer's protocol.

Cell viability and proliferation assays. MB cells transfected with miR-584-5p mimic or target siRNAs were seeded in 96-well plates at a density of 5×10³ for D556Med and DAOY or 10×10³ for D425Med and D458Med per well for 72 hours. Cell viability was assessed using CellTiter-Glo (Promega) or alamarBlue (Thermofisher), and proliferation was assessed using CyQuant Direct Cell Proliferation Assay (Thermofisher).

Statistical analysis. Statistical analysis was done using GraphPad Software, R, or Compusyn.

### Example 2: MiR-584-5p Acts as a Tumor Suppressor and Sensitizes VCR Response In Vivo

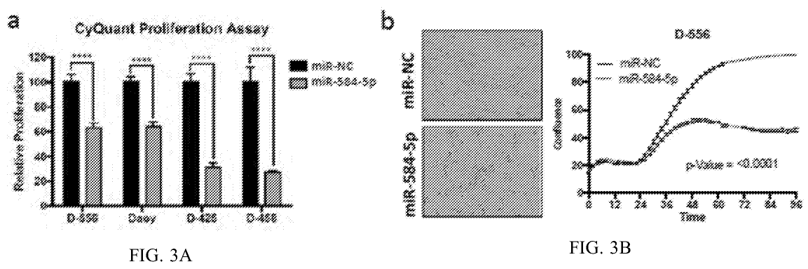

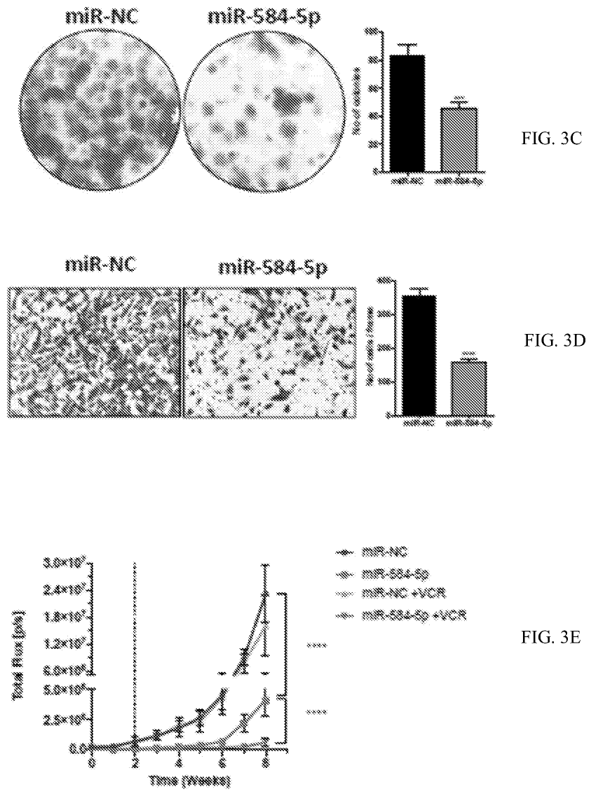

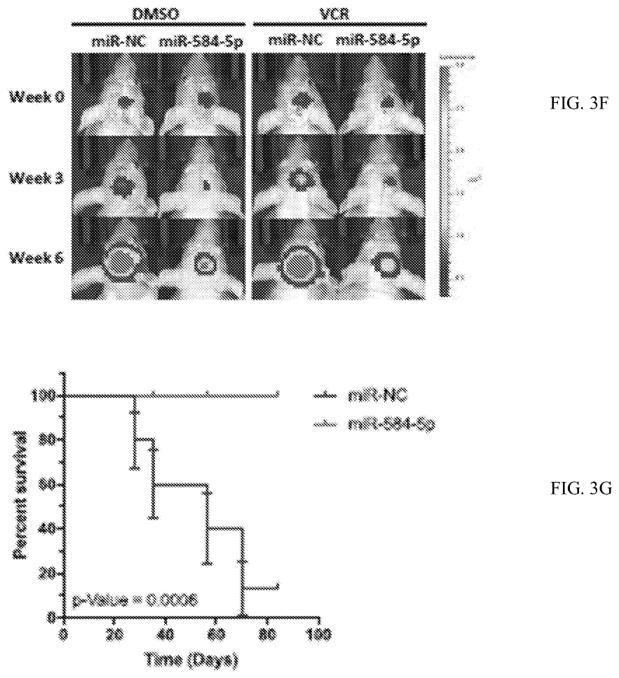

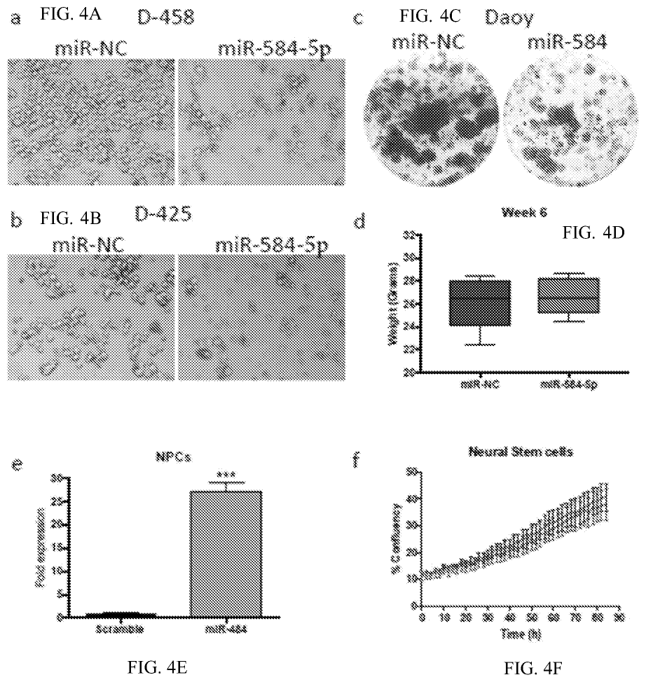

Next, the in vitro results were validated by determining the function of miR-584-5p in MB and by using an in vivo tumor xenograft model to test the efficacy of miR-584-5p-VCR combination therapy. MiR-584-5p reduced the short-term and long-term viability as well as migration of MB cells (FIGS. 3A-D and FIG. 4A-F). In FIG. 3A, cell proliferation was measured by CyQuant Cell Proliferation Assay. The p-values were determined using standard Student t-tests. Error bars represent mean±SEM. In FIG. 3B, cell proliferation was measured using an IncuCyte phase-only processing module. The p-values were determined by the sum-of-squares F test. Error bars represent mean±standard deviation. In FIG. 3C, the bar graph shows number of crystal violet-stained colonies. The p-value was calculated using a standard Student t-test. Error bars represent mean±SEM. In FIG. 3D, the bar graph shows number of migrated cells counted microscopically in at least 10 fields. The p-value was calculated using a standard Student t-test. Error bars represent mean±SEM. In FIG. 4F, The p-value was calculated using a standard Student t-test. Error bars represent mean±SEM. Cell proliferation was measured using an IncuCyte phase-only processing module. ****, p<0.0001.

For in vivo studies, orthotopic intracranial transplantation of the scrambled and miR-584-5p-transfected DAOY cells stably expressing green fluorescent protein (GFP)-luciferase was performed. MiR-584-5p transfectant tumors showed dramatically reduced MB growth in comparison with scrambled-transfectant tumor (FIGS. 3E, F) suggesting that miR-584-5p acts as a potent tumor suppressor in MB. Furthermore, combination therapy of miR-584-5p and a sublethal dose of VCR significantly reduced tumor growth in comparison with either miR-584-5p or VCR alone (FIGS. 3E, F). In FIG. 3E, the dotted line represents the start of VCR IP injections. The p-values were determined by the sum-of-squares F test. Error bars represent mean±SEM. Consistent with that finding, mice treated with miR-584-5p and miR-584-5p-VCR lived significantly longer than the scrambled-transfectant group (FIG. 3G). The p-value was determined using the log-rank (Mantel-Cox) test. Error bars represent SE. ****, p<0.0001; ***, p<0.001.

Animals and intracranial xenografts. Athymic nude mice (nu/nu) were obtained from Harlan, USA. Three- to 5-week-old athymic nude mice (nu/nu) were anesthetized using isoflurane by inhalation. Using a Hamilton syringe, 1 million cells were stereotactically implanted into the right corpus striatum at a depth of 3.5 mm at a point 2.5 mm lateral to the midline and 1.5 mm anterior to the bregma. After the surgery, the animal recovered on a heating pad in a normal mouse cage to maintain its body temperature at 37°-37.2° C. An ocular lubricating ointment (AKORN) was administered until spontaneous blinking resumed. Starting from week 2, control group animals received dimethyl sulfoxide, whereas VCR group animals received 1 mg of VCR (Sigma-Aldrich) per kg of body weight intraperitoneally once per week for 6 weeks. The Xenogen Small-Animal Imaging System was used for subcellular imaging in live mice once per week. Animals were killed, and brains were fixed and analyzed.

Migration and colony formation assays. MB cells were transfected with miR-584-5p, miR-NC, scramble-siRNA, or target-siRNA (Sigma) for 24 hours, harvested, and subjected to long-term clonogenic and migration assays (Rajamanickam, S. et al. Clin Cancer Res 22, 3524-3536, (2016)). For the long-term clonogenic assays, 1,000 cells/well were reseeded in six-well plates for an additional 7-10 days until colonies were visible. Colonies were fixed with 4% paraformaldehyde and stained with 1% crystal violet. For the transwell migration assay, 100,000 cells were reseeded in Corning BioCoat Control Cell Culture Inserts with 8.0-μm PET Membrane in serum-free medium. Cells were allowed to migrate toward complete media for 24 hours before fixation with 4% paraformaldehyde and staining with 1% crystal violet.

Dual luciferase assay. Wild-type or mutant 3′-UTR segments of the eIF4E3 and HDAC1 gene were cloned downstream of the luciferase gene in pGL3-promoter vector at the SacI and SpeI restriction sites. HEK293 cells were cotransfected with Renilla luciferase vector (pRLnull 380) and firefly luciferase vector containing pGL3-wt-eIF4E3; pGL3-mut-eIF4E3; or pGL3-wt-HDAC1, pGL3-mut-HDAC1, or pGL3 overnight and incubated in fresh complete medium for an additional 48 hours after transfection. Cells were then transfected with miRNA mimic (Invitrogen) for 24 hours. Next, cells were harvested with 1× passive lysis buffer (Promega) and luciferase activity was read using GLOMAX 20/20 luminometer (Promega).

### Example 3: MiR-584-5p Inhibits MB Growth and Sensitizes VCR Response by Targeting Genes Associated with Microtubule Dynamics and Translation Initiation

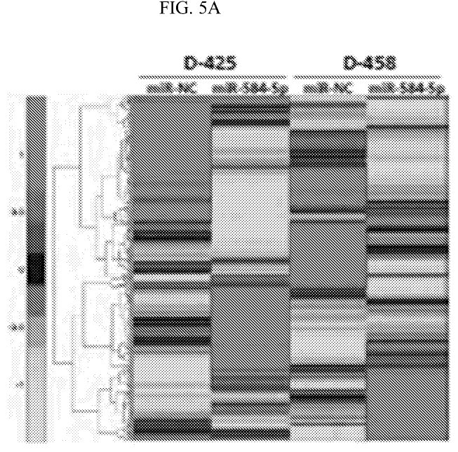

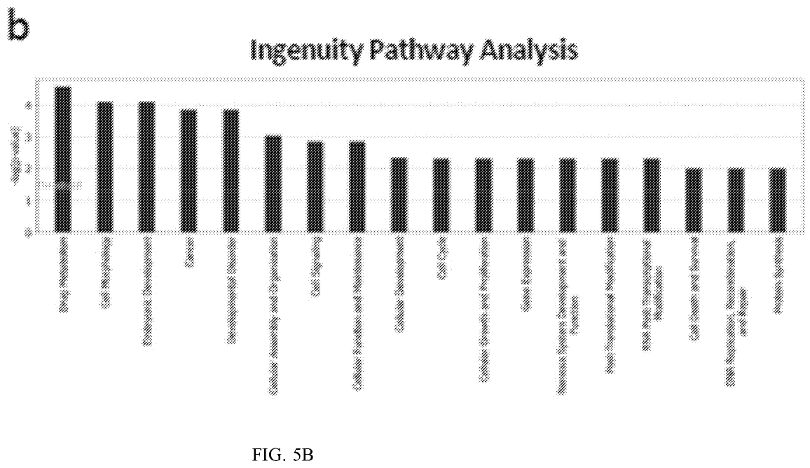

To investigate the mechanism by which miR-584-5p inhibits MB growth and enhances VCR response, gene expression analysis was performed on D458Med and D425Med MB cells overexpressing miR-584-5p mimic. Ingenuity pathway analysis revealed some highly altered downstream biological pathways in miR-584-5p-expressing MB cells: drug metabolism, cellular assembly and organization, nervous system development and function, embryonic development, and translation initiation (FIGS. 5A, B). Data are z-transformed. The cutoff criteria for each gene are fold change >2, sample intensity >10, and p-value <0.05. Examples of genes associated with those biological pathways included HDAC1 and eIF4E3, the most downregulated in the gene expression analysis. Moreover, multiple target prediction algorithms, including Target Scan and Diana micro-T-CDS, predicted that miR-584-5p would target HDAC1 and eIF4E3. To validate the gene expression analysis and target prediction results, it was determined whether miR-584-5p reduced HDAC1/eIF4E3 expression by binding to their 3′-untranslated regions (3′-UTRs). The luciferase activity of a construct containing luciferase gene fused with the 3′-UTR of eIF4E3 or HDAC1 was significantly reduced in MB cells expressing miR-584-5p mimic in comparison with scrambled. In contrast, mutation of miR-584-5p biding sites in 3′-UTR of HDAC1/eIF4E3 resulted in no change in luciferase activity in the presence of miR-584-5p mimic in comparison with scrambled, confirming the interaction between miR-584-5p and predicted binding sites in the 3′-UTR of those genes (FIG. 5C). In FIG. 5C, the inset boxes show miR-584-5p binding sites in 3′-UTRs of eIF4E3 and HDAC1 (wild type); SEQ ID NO: 11 (CCCAGGGAAAAGAAAAACCATA) and SEQ ID NO: 12 (CCCAGGGAAAAGAAGGGTTGCG) represent the EIF4E3 3′UTR sequences; and SEQ ID NO: 13 (CCGTTCTTAACTTTGAACCATAA) and SEQ ID NO: 14 (CCGTTCTTAACTTTGGGTTGCGG) represent the HDAC1 3′UTR sequences. Also shown are the portion of the miR-584-5p sequence (SEQ ID NO: 15; UAUGGUU) that binds to the wild-type 3′UTR and seed sequence mutant (SEQ ID NO: 16; GGGTTGCG, top panel; and SEQ ID NO: 17; GGTTGCGG; bottom panel). Also shown is miR-584-5p seed sequence mutated sequences. Right, relative luciferase activity in HEK 293 cells transfected with miR-NC, miR-584-5p, or miR-584-5p binding site mutant (mutant). Values were normalized to firefly luciferase, which served as an internal control. The p-values were calculated using standard Student t-tests. Error bars represent mean±SEM. To further support that finding, the levels of HDAC1/eIF4E3 in miR-584-5p-overexpressing MB cells was determined. MiR-584-5p overexpression resulted in significantly reduced expression of HDAC1/eIF4E3 at both the transcript and protein levels in all cell lines tested (FIGS. 5D, E). The p-values were calculated using standard Student t-tests. Error bars represent mean±SEM. ****, p<0.0001; ***, p<0.001.

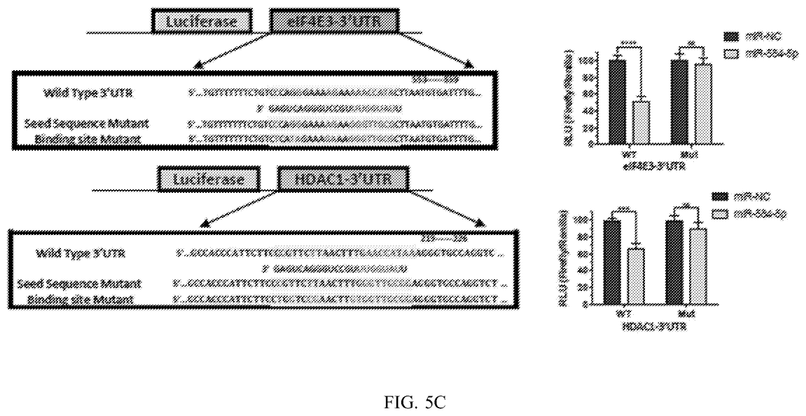

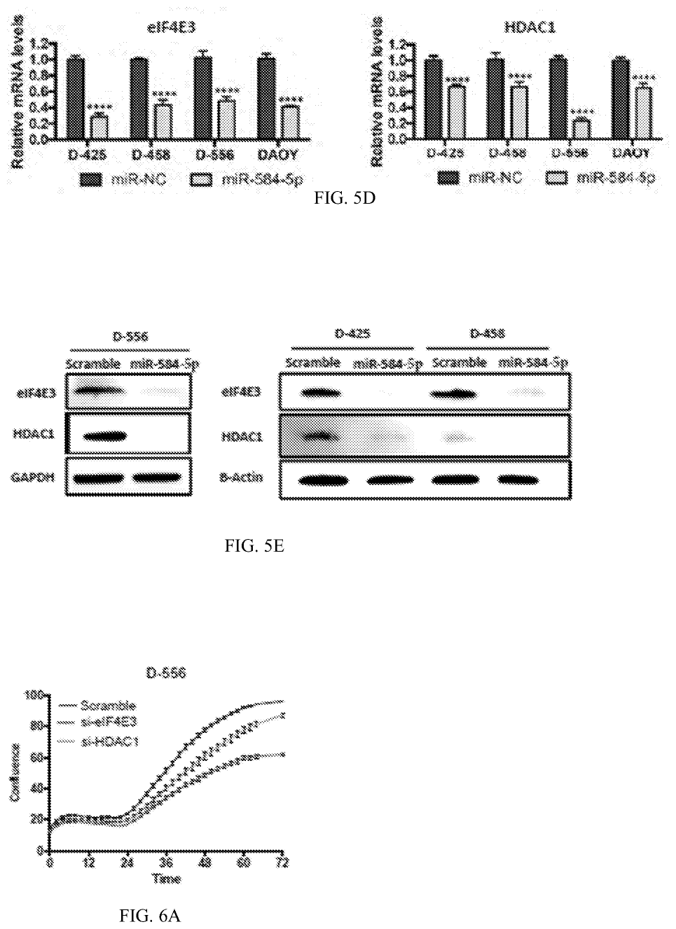

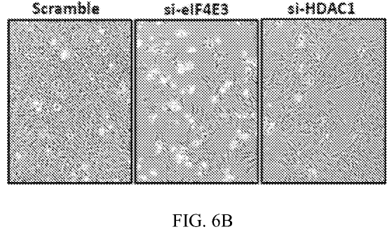

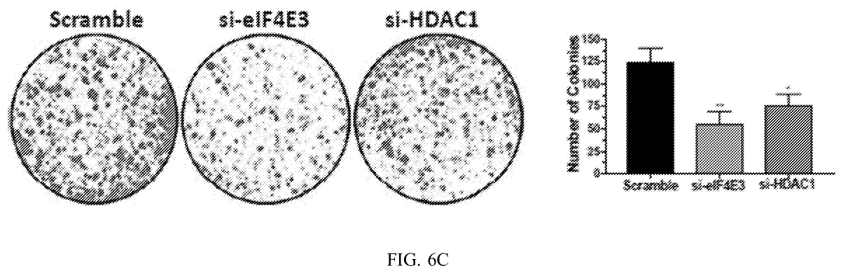

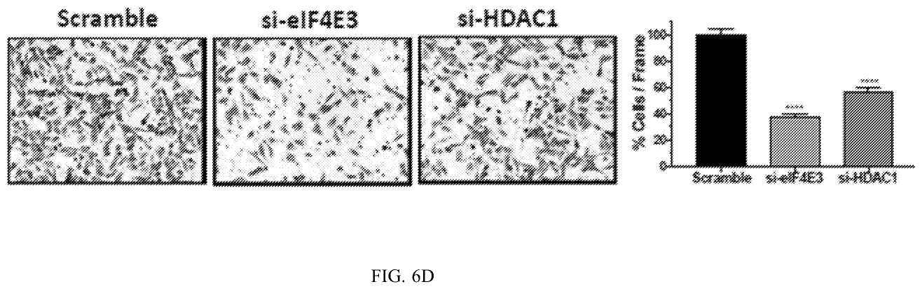

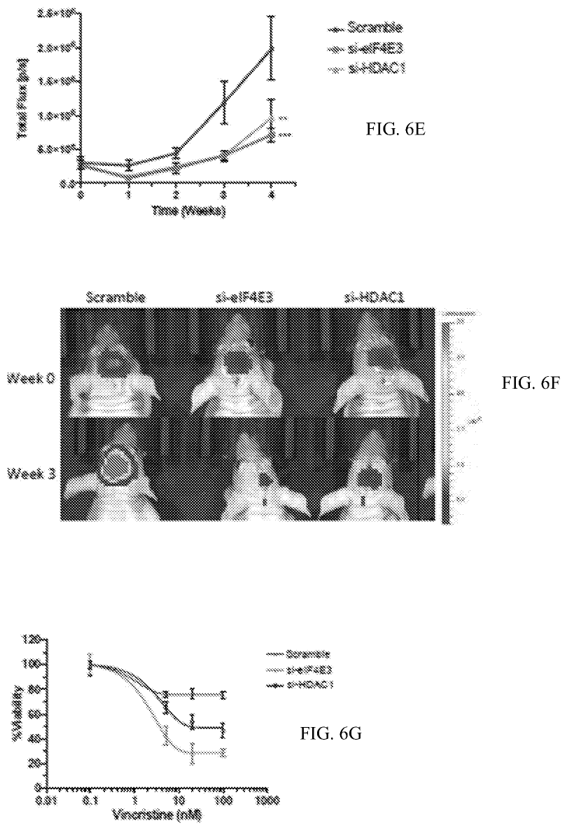

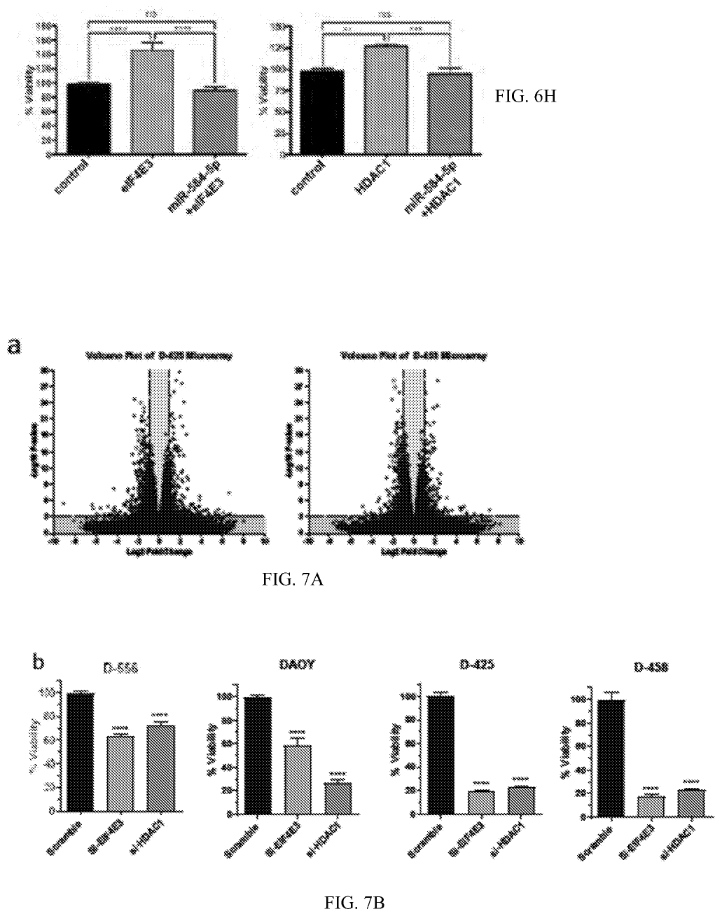

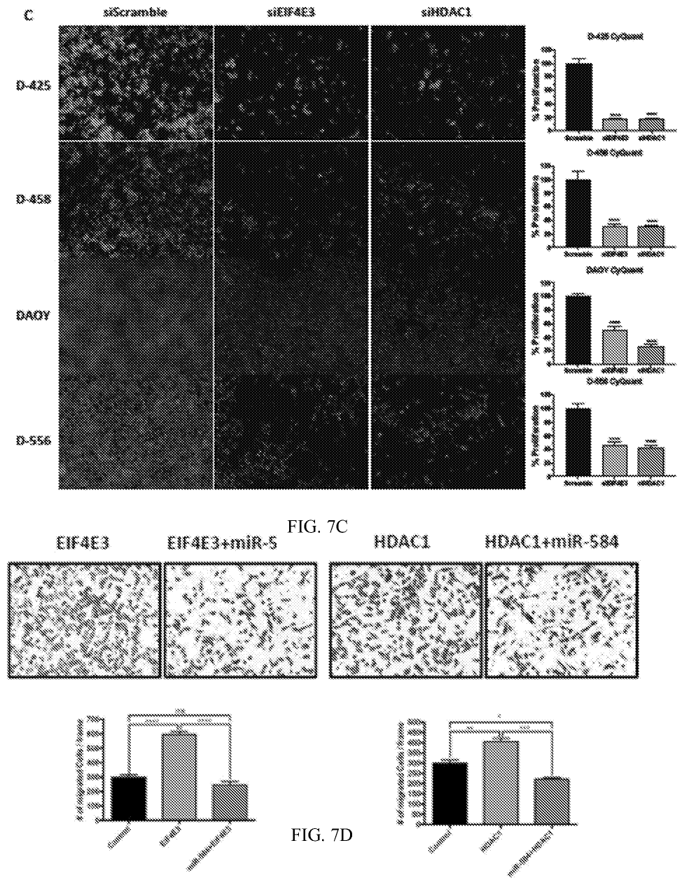

To determine whether miR-584-5p imparts its tumor-suppressing and VCR-sensitizing effect by downregulating HDAC1/eIF4E3 levels in MB, the function of eIF4E3 and HDAC1 in MB was investigated. Silencing HDAC1 or eIF4E3 led to significantly reduced viability, proliferation, and migration of MB cells (FIGS. 6A-D and FIG. 7A-D). In FIG. 6A, cell proliferation was measured using an IncuCyte phase-only processing module. The p-values were determined by the sum-of-squares F test. Error bars represent mean±SD. In FIG. 6C, the bar graph shows crystal violet-stained colonies counted microscopically. The p-values in FIGS. 6C and D were calculated using a standard Student t-test. Error bars represent mean±SEM. In FIG. 7B, cell viability was assessed using alamarBlue cell viability assay. The p-value was calculated using one-way ANOVA followed by Dunnett's multiple-comparisons test. In FIG. 7C, quantification of CyQuant fluorescence signal of D556Med, D425Med, D458Med, and DAOY cells transfected with 50 nM Scramble, eIF4E3, or HDAC1 siRNAs. The p-value was calculated using one-way ANOVA followed by Dunnett's multiple-comparisons test. In FIG. 7D, the bar graphs show viability of D556Med cells transfected with control plasmid or eIF4E3/HDAC1 expression vector or cotransfected with eIF4E3/HDAC1 expression vector and miR-584-5p mimic. The p-value was calculated using one-way ANOVA followed by Sidak's multiple-comparisons test. Error bars represent mean±SEM. ****, p<0.0001; ***, p<0.001; **, p<0.01; *, p<0.05. Furthermore, depletion of HDAC1 or eIF4E3 resulted in significantly reduced MB growth in orthotopic intracranial xenograft models (FIGS. 6E, F). In FIG. 6E, the p-values were determined by the sum-of-squares F test. Error bars represent mean±SEM. Moreover, depletion of HDAC1 or eIF4E3 significantly enhanced the VCR sensitivity of the MB cells (FIG. 6G). Cell viability was assessed using alamarBlue cell viability assay. The p-values were determined by the sum-of-squares F test. Error bars represent mean±SEM. Notably, miR-584-5p rescued the increased long-term viability and migration of MB cells elicited by HDAC1/eIF4E3 overexpression (FIG. 6H). Bar graphs showing viability of D556Med cells transfected with control plasmid or eIF4E3/HDAC1 expression vector or cotransfected with eIF4E3/HDAC1 expression vector and miR-584-5p mimic. The p-value was calculated using one-way analysis of variance (ANOVA) followed by Sidak's multiple-comparisons test. Error bars represent mean±SEM. ****, p<0.0001; ***, p<0.001; **, p<0.01. Taken together, the results clearly indicated that miR-584-5p inhibits MB growth and progression by inhibiting the tumor-promoting function of HDAC1 or eIF4E3.

Gene expression profiling. Total RNA was isolated from D425Med and D458Med cells after miR-584-5p transfection for 48 hours. RNA samples were further processed at the UTHSCSA Genomics Core for gene-expression profiling by using Illumina Human HT-12 v4 Expression BeadChip following the manufacturer's standard protocol. Gene-expression data were quantified and normalized (quantile normalization) using BeadStudio software (Illumina). A Union gene set of 1,152 genes differentially expressed in both D425Med and D458Med cells treated with miR-NC or miR-584-5p mimic for 48 hours was used. Data were z-transformed, and the cutoff criteria for each gene are a fold change >2, sample intensity >10, and p-value <0.05.

The results disclosed herein show that miR-584-5p suppresses MB growth by inhibiting HR-mediated DNA repair. Furthermore, the results show that silencing HDAC1 or eIF4E3 inhibits MB growth and sensitizes IR response by promoting DNA damage and HR-mediated DNA repair. HDACs remove acetyl groups from histones, thereby compacting the chromatin. Therefore, inhibiting HDAC1 by miR-584-5p probably damages DNA in MB cells by allowing genotoxic insults, such as those from chemotherapy drugs and IR, to reach the chromatin. Furthermore, HDAC inhibitors decrease the expression of proteins involved in repairing chemotherapy drug-induced DNA damage (Roos, W. P. & Krumm, A. Nucleic Acids Res 44, 10017-10030, (2016)). For example, MS-275, which inhibits class I HDACs, including HDAC1, decreases RAD51 expression and blocks HR in melanoma cells (Krumm, A. et al. Cancer Res 76, 3067-3077, (2016)). In addition, HDAC1 interacts with proliferating cell nuclear antigen, which is required for HR (Li, J., Holzschu, D. L. & Sugiyama, T. Proc Natl Acad Sci USA 110, 7672-7677, (2013)). Those facts suggest that approaches aimed at inhibiting HDAC1 (such as miR-584-5p) may inhibit MB growth and progression as well as sensitize therapy response by affecting multiple aspects of DNA damage response in MB cells. In addition to HDAC1, miR-584-5p may affect DNA damage and repair in MB by targeting eIF4E3-dependent signaling. Increased expression of eIF4E3 in MB may increase translation of those mRNAs involved in cell survival and DNA damage response. For example, inhibiting eIF4G1 delays resolution of DNA double-strand breaks in breast cancer cells by reducing CHK1, CHK2, and BRCA1 levels (Badura, M., Braunstein, S., Zavadil, J. & Schneider, R. J. Proc Natl Acad Sci USA 109, 18767-18772, (2012)). eIF4E3 interacts with eIF4G1 (Frydryskova, K. et al. BMC Mol Biol 17, 21, (2016)). Furthermore, our results show that miR-584-5p inhibits CHK1 and ATR levels in MB cells.

### Example 4: MiR-584-5p-HDAC1/eIF4E3 Signaling Axis Regulates Cell Cycle Progression of MB Cells

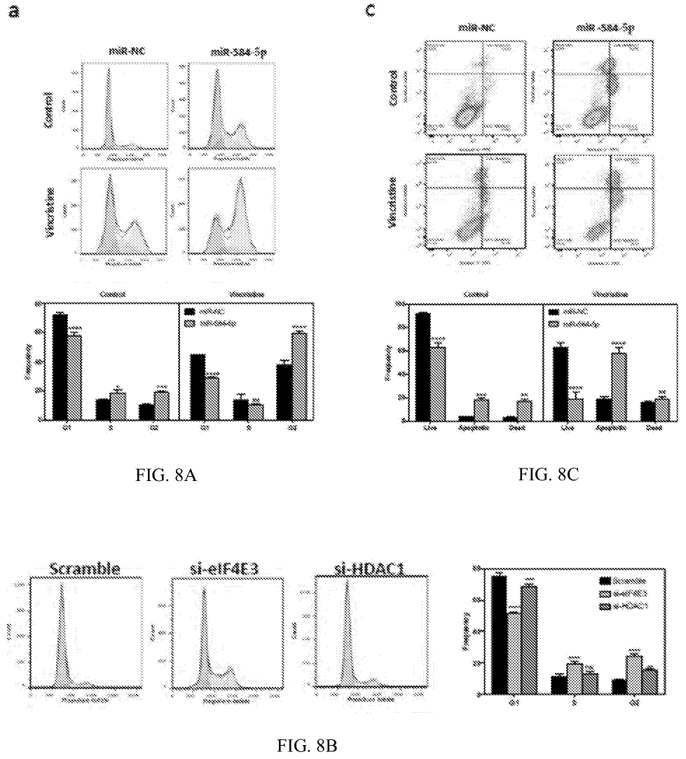

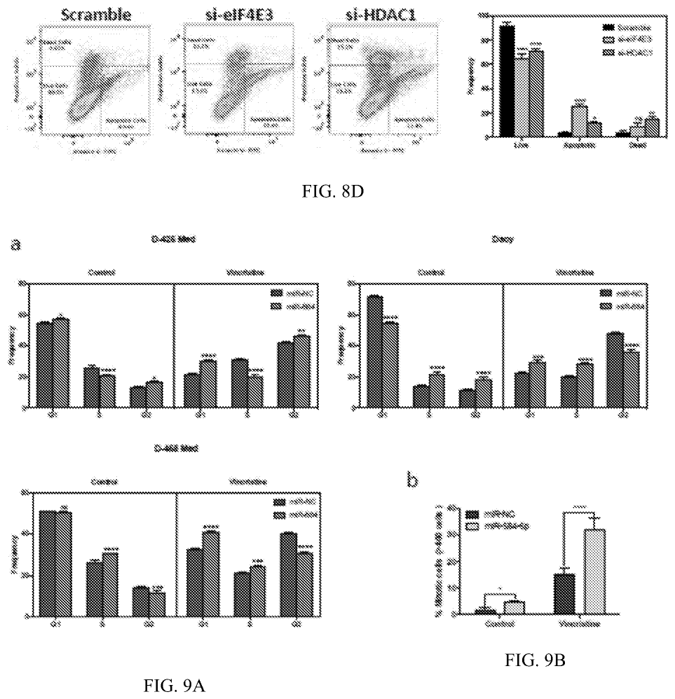

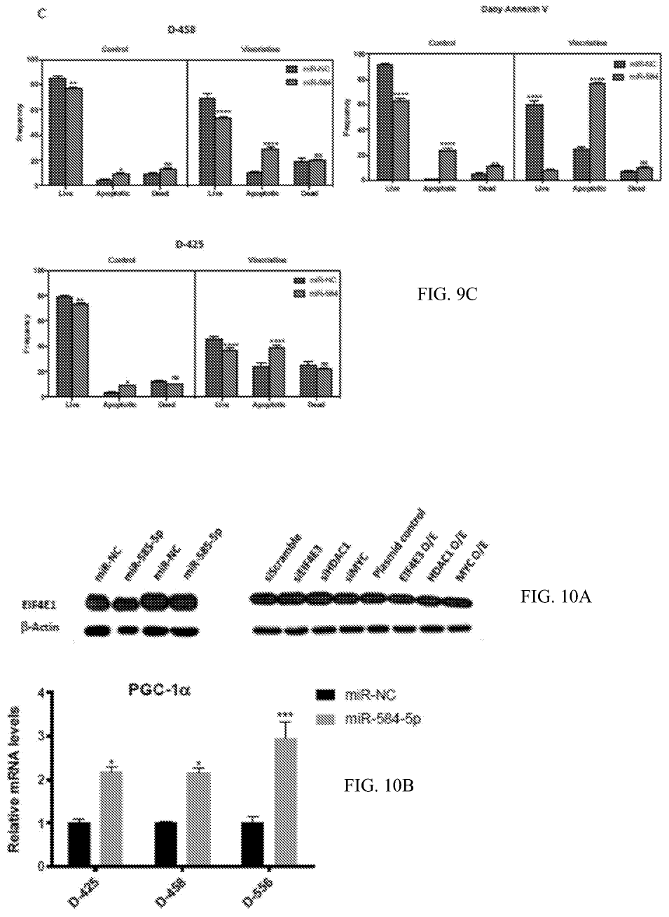

To gain more insight into the tumor-suppressing and VCR-sensitizing activities of miR-584-5p, it was determined how the miR-584-5p mimic affected cell cycle progression of MB cells. MB cells treated with miR-584-5p mimic showed G₂/M cell cycle arrest (FIG. 8A and FIG. 9). Bar graphs show DNA histograms quantified using the FlowJo software. In FIG. 9A, the p-values were determined by two-way ANOVA followed by Tukey's multiple-comparisons test. Error bars represent mean±SEM. In addition, miR-584-5p-VCR combination treatment significantly increased the percentage of cells at G₂/M phase in comparison with either VCR or miR-584-5p alone (FIG. 8A and FIG. 9). In FIG. 9B, the p-values were calculated using standard Student t-tests. Error bars represent mean±SEM. To further confirm those findings, the mitotic cells were counted Immunofluorescence analysis revealed the presence of significantly more mitotic cells in miR-584-5p mimic-transfected MB cells than scrambled-transfected cells (FIG. 9). Those findings may partly explain the sensitizing effect of the miR-584-5p-VCR combination because VCR induces G₂/M phase arrest (George, P., Journey, L. J. & Goldstein, M. N. J Natl Cancer Inst 35, 355-375 (1965)). Next, it was determined whether miR-584-5p's effect on cell cycle progression may be mediated via its target genes eIF4E3/HDAC1. Indeed, silencing either eIF4E3 or HDAC1 resulted in G₂/M phase arrest (FIG. 8B). Bar graphs show dot plots quantified using the FlowJo software. Next, it was tested whether miR-584-5p-induced cell cycle arrest led to apoptosis of MB cells. Annexin V staining followed by fluorescence-activated cell sorter (FACS) analysis revealed that miR-584-5p mimic or eIF4E3/HDAC1 silencing resulted in increased apoptosis of MB cells (FIGS. 8C, D and FIG. 9). In FIG. 8C, the bar graphs reflect DNA histograms quantified using the FlowJo software and in FIG. 8D and FIG. 9C, the bar graphs show dot plots quantified using the FlowJo software. The p-values for panels 8A-D and FIG. 9C were determined by two-way ANOVA followed by Tukey's multiple-comparisons test. Error bars represent mean±SEM. ****, p<0.0001; ***, p<0.001; **, p<0.01; *, p<0.05.

Flow cytometry analysis of cell cycle and apoptosis. MB cells were seeded in six-well plates and transfected with miR-584-5p mimic or target gene siRNAs and incubated for 48 hours. Cells were then treated with VCR or vehicle for 72 hours before being stained with propidium iodide and Annexin V-fluorescein isothiocyanate (FITC) to analyze apoptosis. For cell cycle analysis, treated cells were fixed using 70% ethanol for 24 hours followed by propidium iodide staining. Cells were analyzed using either FACScantoII or LSRII cytometers, and data were quantified using FlowJo 10.2 software.

Immunofluorescence and Western blot analysis Immunofluorescence analyses was carried out on cells fixed in 4% paraformaldehyde (Imam, J. S. et al. PLoS One 7, e52397, (2012)). Western blot analysis was performed on cell extracts from miR-584-5p mimic- or target gene siRNA-transfected MB cell lines in accordance with previously reported studies (Rajamanickam, S. et al. Clin Cancer Res 22, 3524-3536, (2016); and Imam, J. S. et al. PLoS One 7, e52397, (2012). Antibodies against horseradish peroxidase-conjugated β-actin (1:50,000; #A5316), β-tubulin (#T8328), and glyceraldehyde-3-phosphate dehydrogenase (GAPDH) (1:25,000; #G9295) were purchased from Sigma-Aldrich. Antibody against eIF4E3 (1:1000; #17282-1-AP) was purchased from Proteintech. Antibodies against HDAC1 (1:1000; #5356), pCHK1 (1:1000; #2348), and CHK1 (1:1000; #2360) were purchased from Cell Signaling Technology (Danvers, Mass., USA). Antibodies against MYC (1:1000; #Sc-40), BRCA1 (1:1000; #6954), and ATR (1:1000; #sc-515173) were purchased from Santa Cruz. Antibody against 53BP1 (1:300; #A300-272AT) was purchased from Bethyl Laboratories.

The results described herein show that miR-584-5p target gene eIF4E3 supports MB growth and progression. In contrast to the results described herein, Osborne and colleagues showed that eIF4E3 inhibited anchorage-independent growth of NIH3T3 fibroblast and U2OS osteosarcoma cells (Osborne, M. J. et al. Proc Natl Acad Sci USA 110, 3877-3882, (2013)). However, Landon and colleagues later proposed that eIF4E3 can also support cell viability by inducing the expression of pro-proliferative genes (Landon, A. L. et al. Nat Commun 5, 5413, (2014)). Furthermore, eIF4E3 induced n-Myc, whereas eIF4E1 induced c-Myc expression in diffuse large B-cell lymphoma (Landon, A. L. et al. Nat Commun 5, 5413, doi:10.1038/ncomms6413 (2014)). As disclosed herein, the results show that eIF4E3 induces c-Myc protein expression. Translation initiation factors can regulate how any specific mRNA, including oncogenic mRNAs, can be translated. Therefore, eIF4E3 hyperactivation may result in increased abundance and activity of c-Myc, which in turn can play a causal role in cellular transformation of neural precursor cells and support MB growth by promoting cell proliferation, blocking apoptosis, or both. Supporting that notion, c-Myc transcriptionally activates Mc1-1 (Labisso, W. L. et al. Cell Cycle 11, 1593-1602, (2012)), an anti-apoptotic protein that is often upregulated in cancers and promotes cancer cell survival.

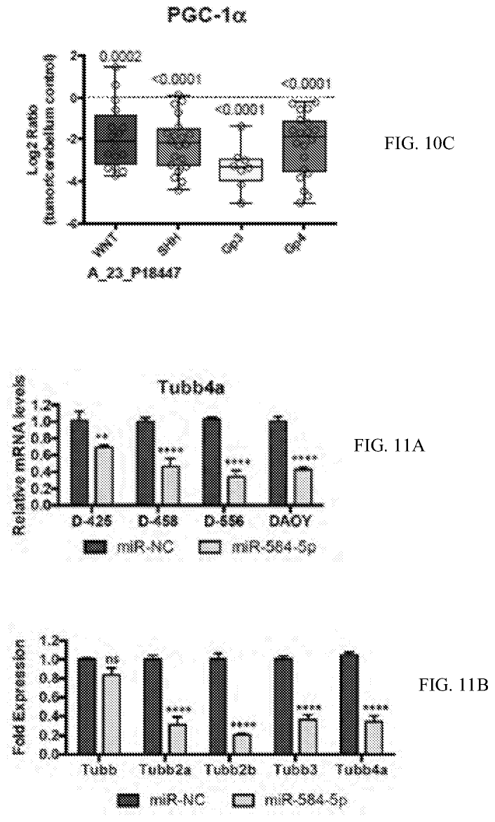

In addition to eIF4E3, the results disclosed herein also show that HDAC1 promotes MB growth and that miR-584-5p inhibits MB growth and progression by inhibiting HDAC1 expression. A pan HDAC inhibitor inhibited c-Myc-amplified MB growth by inhibiting FOXO1 (Ecker, J. et al. Acta Neuropathol Commun 3, 22, (2015)). However, the gene expression analysis showed that FOXO1 levels did not change in miR-584-5p-treated MB cells, suggesting that factors other than FOXO1 mediate HDAC1's protumorigenic function in MB. One such gene is peroxisome proliferator-activated receptor gamma coactivator 1-α, which codes for PGC-1α protein, which HDAC1 inhibits in brown adipocytes (Li, F. et al. J Biol Chem 291, 4523-4536, (2016)). The results disclosed herein reveal that PGC-1α expression is increased in miR-584-5p-treated MB cells (FIG. 10). Furthermore, we show significantly lower levels of PGC-1α in MB patients than in normal cerebellar tissues (FIG. 10), suggesting that PGC-1α may act as a tumor suppressor in MB and that altering HDAC1-PGC-1α signaling may partly mediate miR-584-5p's tumor-suppressing action. The data in FIG. 10 represent Log 2 ratio tumor/cerebellum control. Data mined from GSE28245. ***, p<0.001; ** , p<0.01; * , p<0.05.

PGC-1α inhibits prostate cancer growth and metastasis by increasing mitochondrial oxidative processes and by decreasing aerobic glycolysis (Torrano, V. et al. Nat Cell Biol 18, 645-656, (2016)). Because MB cells, like neural precursor cells, resort to increased aerobic glycolysis to meet their high energy demand (Tech, K. & Gershon, T. R. Transl Pediatr 4, 12-19, (2015), this study suggests that miR-584-5p may disrupt the metabolic program to inhibit MB growth and progression. Supporting that notion, the results described herein show that miR-584-5p inhibits expression of c-Myc, which upregulates glycolysis (and therefore directly contributes to the Warburg effect) in several cancers (Miller, D. M., Thomas, S. D., Islam, A., Muench, D. & Sedoris, K. Clin Cancer Res 18, 5546-5553, (2012)). Furthermore, translation initiation factors coordinate the translation program in response to specific oxygen stimulus (Koumenis, C. et al. Mol Cell Biol 22, 7405-7416 (2002)). For example, hypoxia induces the eIF4E2-eIF4A-eIF4G3-dependent protein synthesis pathway in cells (Koumenis, C. et al. Mol Cell Biol 22, 7405-7416 (2002)). eIF4E3 physically interacts with eIF4A (Landon, A. L. et al. Nat Commun 5, 5413, (2014)). Because increased glycolysis is a hallmark of hypoxic tumors and hypoxia promotes MB stem cell survival and expansion (Fan, X. & Eberhart, C. G. J Clin Oncol 26, 2821-2827, (2008)), it is possible that in addition to HDAC1, miR-584-5p affects MB's metabolic program by inhibiting eIF4E3-dependent protein synthesis.

### Example 5: MiR-584-5p-eIF4E3/HDAC1 Signaling Axis Regulates Microtubule Dynamics in MB Cells

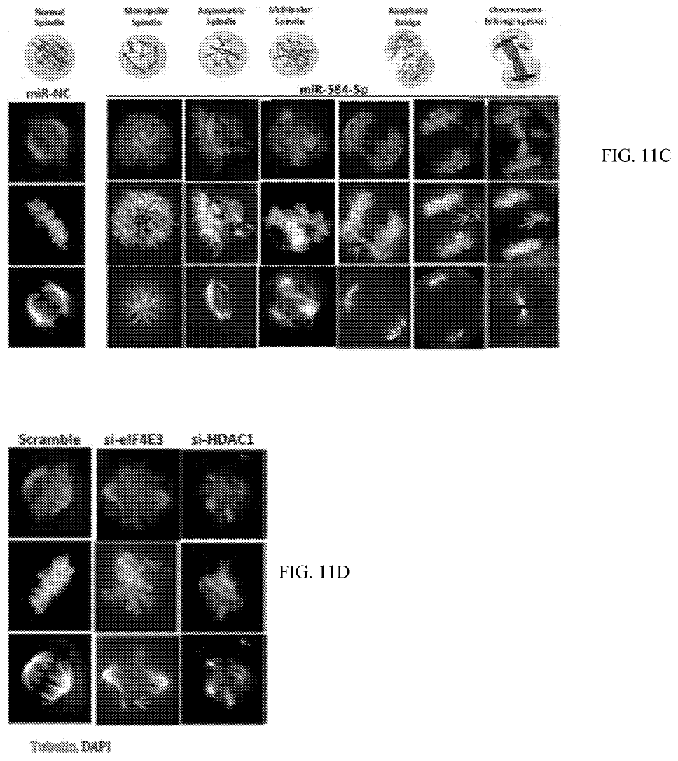

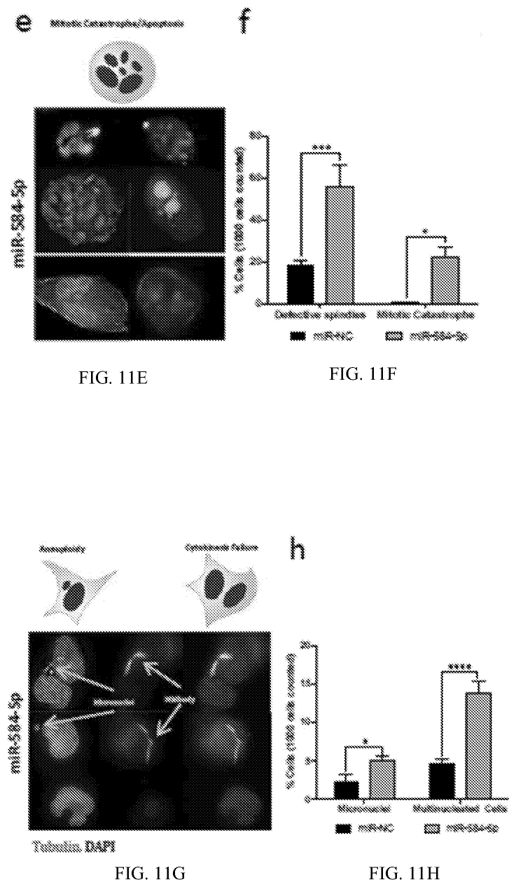

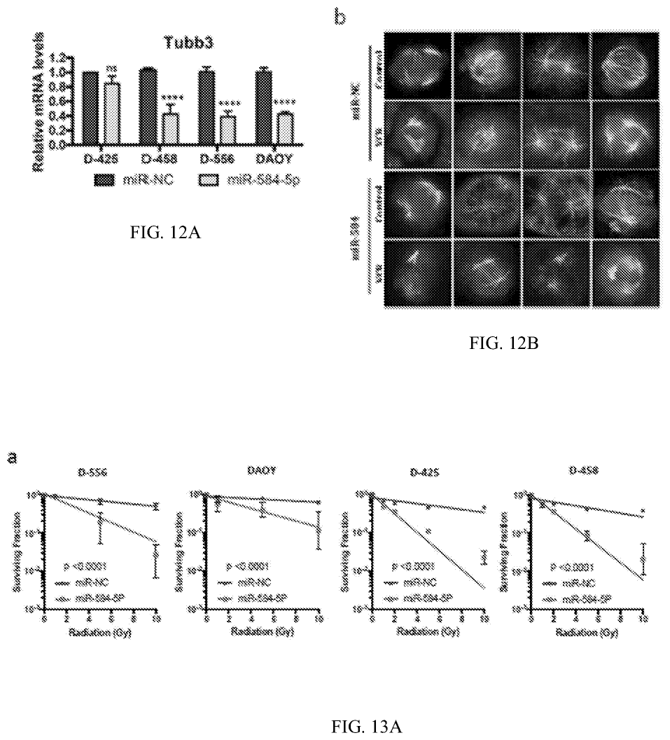

VCR disrupts the integrity of the mitotic spindle and destabilizes microtubules (George, P., Journey, L. J. & Goldstein, M. N. J Natl Cancer Inst 35, 355-375 (1965)). Therefore, it was tested whether miR-584-5p and its target genes might affect microtubule dynamics. First, expression of tubulins was deteremined because VCR resistance has been associated with altered expression of tubulin isotypes (Sirotnak, F. M., Danenberg, K. D., Chen, J., Fritz, F. & Danenberg, P. V. Biochem Biophys Res Commun 269, 21-24, (2000)). The gene expression analysis revealed that TUBB4, a brain-specific β-tubulin, was among the highly downregulated genes, which real-time PCR analysis further confirmed (FIG. 11A and FIG. 12). In FIG. 12A, the p-values were calculated using standard Student t-tests. Error bars represent mean±SEM. ****, p<0.0001. In addition to TUBB4, miR-584-5p-overexpressing cells consistently exhibited significantly reduced transcript levels of TUBB3, TUBB2a, and TUBB2b (FIG. 11B). The p-values were calculated using standard Student t-tests. Error bars represent mean±SEM. To further test how miR-584-5p and its target genes affect spindle integrity, immunofluorescence analysis was performed on miR-584-5p-overexpressing or eIF4E3/HDAC1-silenced MB cells. MB cells transfected with miR-584-5p mimic exhibited a variety of mitotic spindle defects, including monopolar, asymmetric, tripolar, and tetrapolar spindles as well as anaphase bridges (FIGS. 11C, F and FIG. 12). In FIG. 11F, the p-values were calculated using standard Student t-tests. Error bars represent mean±SEM. Similarly, depleting eIF4E3 or HDAC1 resulted in more multipolar as well as collapsed spindles in MB cells (FIG. 11D). Those findings suggested that eIF4E3 and HDAC1 are important mediators of miR-584-5p regulation of microtubule dynamics. In FIG. 11H, The p-values were calculated using standard Student t-tests. Error bars represent mean±SEM. ****, p<0.0001; ***, p<0.001; **, p<0.01; *, p<0.05. Quantitative real-time PCR. Total RNA was extracted using miRNAeasy kit (Qiagen). Reverse transcription was done using either miScript II RT Kit (Qiagen) or TaqMan microRNA reverse transcription (Thermofisher). RT-qPCR was done using SYBR green (Qiagen) or TaqMan microRNA Control Assays according to manufacturer's protocols.

The results disclosed herein show that the miR-584-5p-eIF4E3/HDAC1 signaling axis regulates VCR sensitivity by regulating microtubule dynamics in MB. eIF4E3 and HDAC1 may affect the dynamics of microtubules by directly interacting with them. Supporting that notion, HDAC1 forms a complex with microtubules in pathological conditions associated with axonal damage (Kim, J. Y. & Casaccia, P. Cell Cycle 9, 3680-3684, (2010)). Though no direct evidence exists that eIF4E3 interacts with microtubules, translation initiation factors including eIF3, eIF4E, and eIG4G co-fractionate with microtubules (Willett, M., Brocard, M., Davide, A. & Morley, S. J. Biochem J438, 217-227, (2011)). In addition, miR-584-5p may affect microtubule organization/polymerization by inhibiting eIF4E3-c-Myc signaling because c-Myc interacts with α-tubulin and polymerized microtubules (Alexandrova, N. et al. Mol Cell Biol 15, 5188-5195 (1995)). Furthermore, a transcriptionally inactive form of myc called myc-nick promotes α-tubulin acetylation and microtubule stabilization (Conacci-Sorrell, M., Ngouenet, C., Anderson, S., Brabletz, T. & Eisenman, R. N. Genes Dev 28, 689-707, (2014)).

Further, these results demonatrate that miR-584-5p-HDAC1/eIF4E3 is a new signaling axis that regulates growth, progression, and therapy response in MB. The results disclosed herein are the first study that implicates miR-584-5p, eIF4E3, and HDAC1 in regulating microtubule dynamics and DNA damage response in MB. Furthermore, the VCR- and IR-sensitizing effects of miR-584-5p mimic and eIF4E3/HDAC1 inhibition serve as a strong rationale for developing miR-584-5p mimic or inhibitors of eIF4E3/HDAC1 as therapeutic regimens for treating MB in general and c-Myc-amplified MB in particular.

### Example 6: MiR-584-5p Induces DNA Damage and Sensitizes Radiation Response in MB Cells

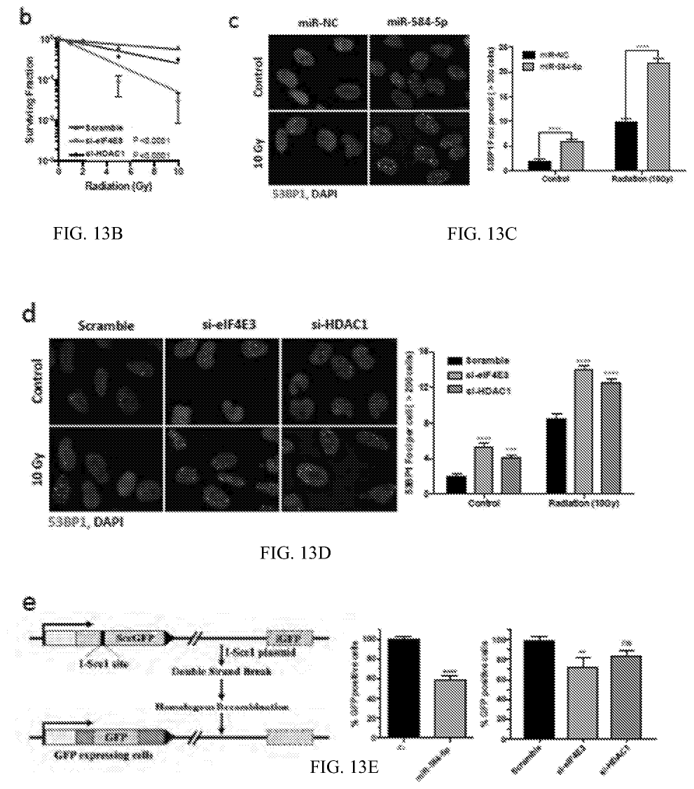

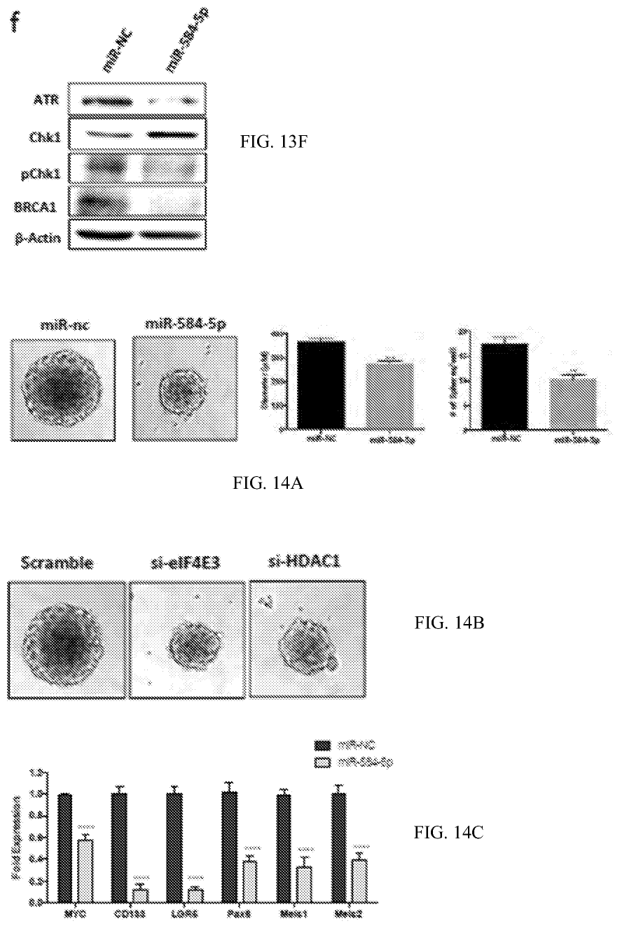

VCR is routinely used during and after radiation therapy to treat MB patients (Packer, R. J. et al. J Neurosurg 74, 433-440, (1991)), mainly because microtubule-targeting agents such as VCR enhances the toxic effects of DNA-damaging agents, including radiation (Poruchynsky, M. S. et al. Proc Natl Acad Sci USA 112, 1571-1576, (2015); and Oehler, C. et al. Neuro Oncol 13, 1000-1010, doi:10.1093/neuonc/nor069 (2011)). Moreover, cells in G₂/M phase are the most sensitive to IR (Pawlik, T. M. & Keyomarsi, K. Int J Radiat Oncol Biol Phys 59, 928-942, (2004)). The results described herein showing miR-584-5p mimic causing mitotic defects and G₂/M arrest suggest that miR-584-5p may also regulate radiosensitivity of MB cells. Indeed, miR-584-5p mimic-transfected MB cells were significantly more sensitive (10-fold) to IR than scrambled-transfected cells (FIG. 13A). miR-NC or miR-584-5p mimic-transfected cells were treated with IR for 24 hours before being subjected to alamarBlue cell viability assay. All values were normalized to 0 Gy. The p-values were determined by the sum-of-squares F test. Error bars represent mean±SEM. Next, it was determined whether eIF4E3 or HDAC1 may mediate miR-584-5p's IR response. Indeed, silencing eIF4E3 or HDAC1 resulted in significantly increased IR response in MB cells (FIG. 13B). siRNA-transfected cells were treated with IR for 24 hours before being subjected to alamarBlue cell viability assay. All values were normalized to 0 Gy. The p-values were determined by the sum-of-squares F test. Error bars represent mean±SEM.

To understand how miR-584-5p and its target genes may make IR more effective, how miR-584-5p or silencing eIF4E3/HDAC1 affected DNA damage response in MB cells as IR damages DNA was assessed. For these experiments, the amount of DNA strand breaks in miR-584-5p mimic or eIF4E3/HDAC1-silenced MB cells was assessed by determining the levels of p53 binding protein (53BP1) nuclear foci (Panier, S. & Boulton, S. J. Nature Reviews Molecular Cell Biology 15, 7-18, (2014)) Immunofluorescence analysis showed significantly more 53BP1 foci in MB cells transfected with miR-584-5p mimic or eIF4E3/HDAC1-small interfering RNA (siRNA) than in scrambled-transfected MB cells (FIGS. 13C, D). Bar graphs next to images show average number of 53BP1 foci/cell. The p-values were determined by two-way ANOVA followed by Sidak's multiple-comparisons test. Error bars represent mean±SEM. Because miR-584-5p induces apoptosis, it was reasoned that DNA strand breaks induced by miR-584-5p and eIF4E3/HDAC1 silencing may not be properly repaired. To confirm those findings, a functional assay was performed to monitor homologous recombination (HR) because HR-dependent events repair IR-induced double-strand breaks in G₂ phase. The ISceI-based DR-GFP reporter assay, which measures the frequency of double-strand break repair by HR (Gunn, A., Bennardo, N., Cheng, A. & Stark, J. M. Journal of Biological Chemistry 286, 42470-42482, (2011); Pierce, A. J., Johnson, R. D., Thompson, L. H. & Jasin, M. Genes & Development 13, 2633-2638, (1999); and Stark, J. M., Pierce, A. J., Oh, J., Pastink, A. & Jasin, M. Molecular and Cellular Biology 24, 9305-9316, (2004)), was used. FACS analysis showed significantly fewer GFP-positive cells in cells transfected with miR-584-5p mimic or eIF4E3-siRNA (but not HDAC1-siRNA) that were stably expressing DR-GFP reporter (FIG. 13E), suggesting that miR-584-5p inhibits, whereas eIF4E3 supports, MB cells' ability to repair DNA. U20S-DR-GFP cells were transfected with miR-NC or miR-584-5p or scramble-siRNA or eIF4E3/HDAC1-siRNAs followed by transfection with pCAGGS vector with ISceI/GFP. I-SceI expression leads to double-strand breaks that HR repairs by using the wild-type GFP, resulting in GFP⁺ cells. The experiment was performed in triplicate along with appropriate controls. The p-values were calculated using either Student t-tests (mimic) or one-way ANOVA followed by Dunnett's multiple-comparisons test (siRNAs). Error bars represent mean±SEM. For FIG. 13, ****, p<0.0001; ***, p<0.001; **, p<0.01; *, p<0.05.

### Example 7: MiR-584-5p Inhibits Checkpoint Functions and Induces Mitotic Catastrophe

Having shown that miR-584-5p induces DNA damage as well as induces G₂/M arrest and mitotic defects, the next set of experiments were carried out to understand the molecular mechanism by which miR-584-5p may mediate those events. To explore that, the status of checkpoint proteins/mediators that regulate the cell cycle progression and mitotic entry to prevent the segregation of damaged chromosomes was examined. MiR-584-5p mimic-transfected MB cells showed significantly lower levels of activated CHK1 and ATR, which play important roles in G₂/M checkpoint control, as well as BRCA1, which affects how long the G₂/M checkpoint phase lasts. Those findings, together with the results described herein, suggest that miR-584-5p mimic-treated cells with unrepaired DNA damage enter mitosis. There, they incur miR-584-5p-induced spindle defects, resulting in mitotic catastrophe, an event that precedes cell death (FIG. 13F). Moreover, mitotic entry with unrepaired DNA as well as impaired spindle formation results in mitotic catastrophe. Supporting that finding, the immunofluorescence analysis of miR-584-5p-overexpressing MB cells showed many cells with multipolar metaphase/anaphase, spindles with unorganized poles, micronuclei, and aneuploidy-well-established effects and outcomes of induced mitotic catastrophe. In addition to those defects, it was observed that miR-584-5p mimic-treated MB cells in which cytokinesis failed, resulting in giant cells with abnormal nuclei. That effect may be due to prolonged mitotic delay leading to mitotic slippage, in which cells exit mitosis and form multinucleated cells in G₁ phase (FIGS. 11E-H).

### Example 8: MiR-584-5p Inhibits MB Stem Cell Proliferation

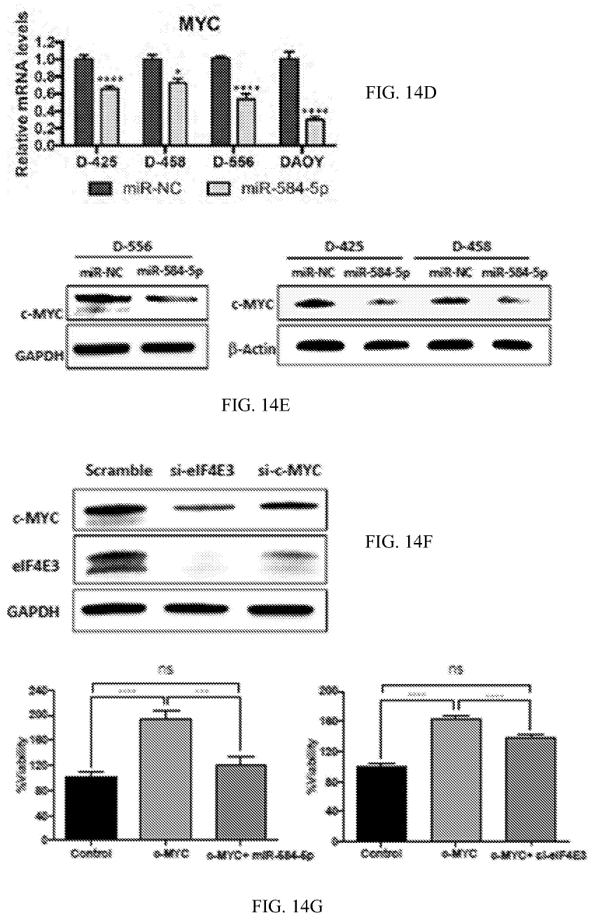

Several research groups have posited the presence of tumor-initiating cells in MBs (Manoranjan, B. et al. Pediatr Res 71, 516-522, (2012)). Moreover, the tumor-initiating cells in general have not responded to radiation and chemotherapy drugs (Manoranj an, B. et al. Pediatr Res 71, 516-522, (2012)). Therefore, it was tested whether miR-584 may inhibit MB tumor-initiating cell proliferation. Consistent with the characteristics of tumor-initiating cells, MB cells cultured in serum-free stem cell media formed tumor spheres that were serially passaged for many cycles (FIG. 14A). Bar graphs show number and size of medullospheres obtained from D556Med cells transfected with miR-NC or miR-584-5p mimic. The p-value was calculated using a standard Student t-test. Error bars represent mean±SEM. MiR-584-5p mimic-transfected MB cells formed significantly smaller and fewer spheres. Silencing miR-584-5p target genes eIF4E3 and HDAC1 also inhibited MB stem cell proliferation (FIG. 14B). Consistent with that finding, levels of several neural stem cell markers reportedly enriched in MB stem cells were significantly reduced in miR-584-5p mimic-transfected cells (FIG. 14C). The p-value was calculated using one-way ANOVA followed by Sidak's multiple-comparisons test. Error bars represent mean±SEM. These findings are highly significant because, this study is one of the first to propose a role for miR-584-5p and its target genes in regulating MB stem cell proliferation.

### Example 9: MiR-584-5p-eIF4E3 Signaling Cascade Regulates c-Myc Levels in MB

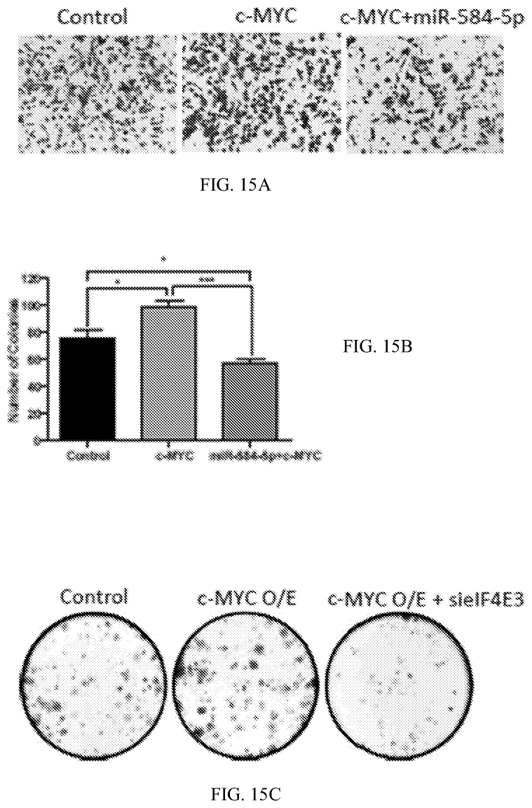

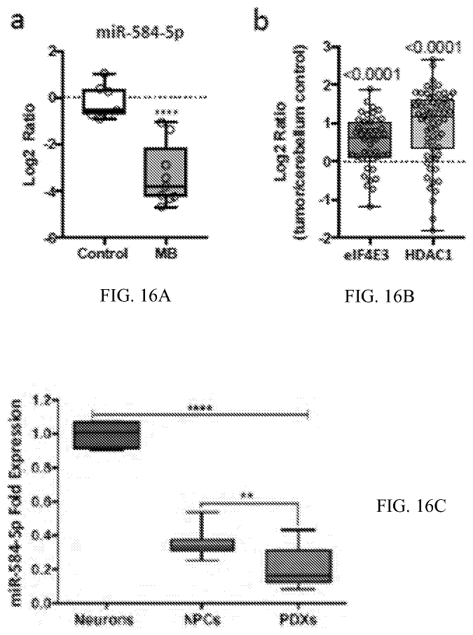

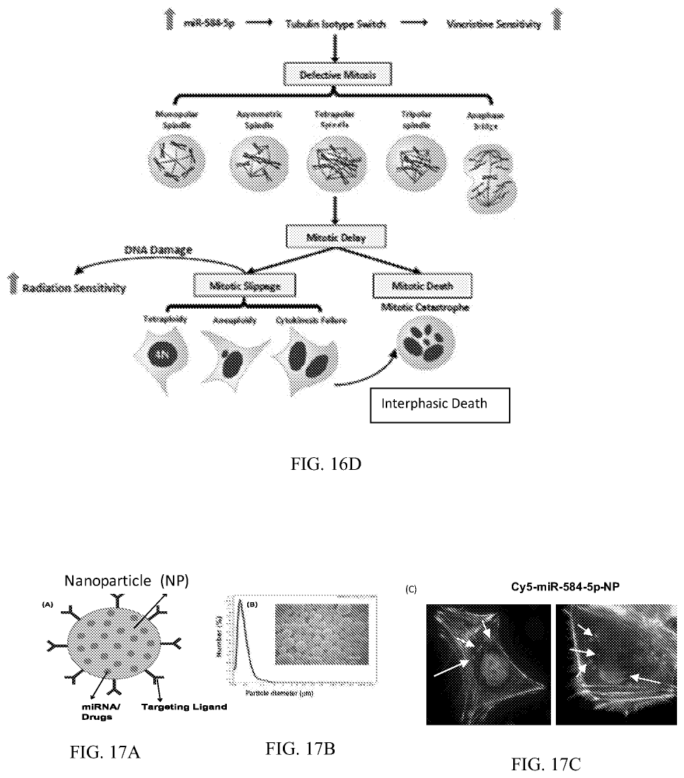

The results described herein showed that miR-584-5p overexpression or depletion of its target genes, eIF4E3/HDAC1, resulted in reduced growth/invasion and VCR/IR sensitization of c-Myc-amplified MB cells. Because c-Myc supports cancer stem cell growth, promotes radioresistance (Gravina, G. L. et al. Radiat Res 185, 411-422, (2016)), and has elevated levels in VCR-resistant cancer cells (He, Y., Zhang, J., Zhang, J. & Yuan, Y. Chin Med J (Engl) 113, 848-851 (2000)), it was tested whether miR-584-5p and its target genes may regulate c-Myc-amplified MB growth and therapy sensitization by targeting c-Myc levels/functions. Indeed, c-Myc-amplified MB cells transfected with miR-584-5p mimic showed significantly reduced expression of c-Myc (FIGS. 14D, E). In FIG. 14D, the p-values were calculated using standard Student t-tests. Error bars represent mean±SEM. Because no predicted miR-584-5p binding site exists in the c-Myc 3′-UTR, miR-584-5p probably regulates c-Myc levels indirectly. To test that possibility, it was assessed whether miR-584-5p target genes may regulate c-Myc levels in MB. Moreover, eIF4E1 (not eIF4E3) induced c-Myc protein expression in diffuse B-cell lymphoma cells (Landon, A. L. et al. Nat Commun 5, 5413, (2014)). Surprisingly, the results revealed that depletion of eIF4E3 significantly reduced c-Myc protein levels in MB cells. However, silencing HDAC1 did not affect c-Myc levels in MB (FIG. 14F). Next, it was tested whether c-Myc may play a role in mediating protumorigenic function of eIF4E3 and whether miR-584-5p inhibits MB growth in part by targeting the eIF4E3-c-Myc signaling cascade. MB cells transfected with c-Myc expression vector followed by eIF4E3 siRNA rescued the growth-promoting effect of c-Myc overexpression (FIG. 14G). Bar graphs show percentage of cell viability in control vector, c-Myc expression vector, or miR-584-5p mimic plus c-Myc expression vector or eIF4E3-siRNA plus c-Myc expression vector-transfected D556Med cells. Cell viability was assessed using alamarBlue. The p-value was calculated using one-way ANOVA followed by Sidak's multiple-comparisons test. Error bars represent mean±SEM. ****, p<0.0001; ***, p<0.001; **, p<0.01; *, p<0.05. Furthermore, c-Myc overexpression rescued the effects of miR-584-5p on MB growth and progression (FIG. 14G and FIG. 15). In FIG. 16C, the clonogenic assay shows number of crystal violet-stained colonies in control plasmid, c-Myc overexpression plasmid or c-Myc overexpression plasmid+eIF4E3 siRNA transfected D556Med cells. ***, p<0.001; **, p<0.01; *, p<0.05. These results suggest that eIF4E3 and c-Myc constitute a positive feedback loop to support MB growth and that miR-584-5p plays an important role in regulating the eIF4E3-c-Myc oncogenic axis in MB.

### Example 10: MiR-584-5p Expression is Significantly Lower in MB

Next, it was determined whether miR-584-5p and its target genes, eIF4E3 and HDAC1, show reciprocal expression pattern in MB. Meta-analysis of a data set from GSE42657 and GSE28245 showed that miR-584-5p expression is significantly lower, whereas eIF4E3 and HDAC1 are highly overexpressed in MB in comparison with normal control (FIGS. 16A, B). In FIG. 16A, data represent Log 2 ratio tumor/cerebellum control. Data mined from GSE42657. In FIG. 16B, data represent Log 2 ratio tumor/cerebellum control. Data mined from GSE28245. To further confirm those results, miR-584-5p expression in MB patient-derived xenografts was examined. The expression level of miR-584-5p was significantly lower in MB patient-derived xenografts than in induced pluripotent stem cell (iPSC)-derived neural progenitor cells and differentiated neurons (FIG. 16C). The p-values were calculated using one-way ANOVA followed by Sidak's multiple-comparisons test. Error bars represent mean±SEM. To further confirm the specificity of miR-584-5p-eIF4E3 signaling axis in MB, the expression of eIF4E1, which acts as an oncogene in several cancers (Bhat, M. et al. Nat Rev Drug Discov 14, 261-278, (2015)), was tested in miR-584-5p treated cells. The results showed that eIF4E1 levels did not show any change in the MB cells overexpressing miR-584-5p (FIG. 10). Furthermore, depletion or overexpression of eIF4E3 didn't alter eIF4E1 levels suggesting that loss of eIF4E3 was not compensated by eIF4E1 in miR-584-5p overexpressing MB cells (FIG. 10). In addition to eIF4E3, silencing or overexpression of HDAC1 or c-Myc didn't result in altered expression of eIF4E1 (FIG. 10).

### Example 11: MiR-584-5p Conjugated with Nanoparticles

Nanoparticle-based approaches can be used to encapsulate miR-584-5p to a nanoparticle to efficiently deliver miR-584-5p across the blood-brain barrier. FIG. 17 shows that miR-584-5p can be encapsulated by nanoparticles. FIG. 17C shows that cy5-labeled miR-584-5p loaded PLGA NPs are internalized into DAOY cells. Poly(lactic-co-glycolic acid (PLGA)-based nanoparticles can be used as a drug delivery system. PLGA facilitates sustained release of therapeutic reagents over a month and can entrap both water-soluble and -insoluble molecules for sustained delivery. In addition, PLGA-NPs are biocompatible. Ligands and antibodies can also be conjugated to PLGA to enhance delivery of miR-584-5p across the blood brain barrier. For example, PLGA can be modified such that one or more ligands can be anchored to the surface of the nanoparticle to facilitate delivery across blood-brain barrier. Examples of ligands include glyco-heptapeptide (g7) which can comprise a modification of which has been shown to enhance cellular uptake across the blood brain barrier (Tosi, G., et al. Curr Med Chem, 2013. 20(17): p. 2212-25). The modification may include substituting one or more amino acids to remove any potential opioid activity without affecting its blood-brain barrier crossing ability. PLGA can also be modified with an antibody against one or more protein that are highly expressed in medulloblastoma compared to normal cerebellum. Examples of proteins include CD24, which is highly expressed in medulloblastoma compared to normal cerebellum (Robson, J. P., et al., PLoS One, 2019. 14(1): p. e0210665).

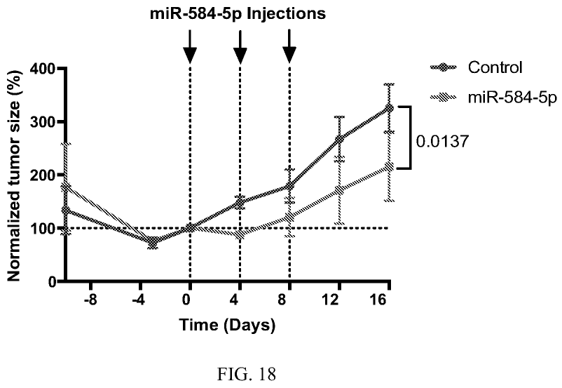

In addition to PLGA, miR-584-5p can be conjugated to a lipid nanoparticle. Examples of lipid nanoparticles may include neutral lipid formulation or intralipid. FIG. 18 shows that the systemic delivery of a miR-584-5p mimic inhibits medulloblastoma growth when miR-584-5p is conjugated with a lipid nanoparticle.

In addition, miR-584-5p can be made more stable by substituting 1 or 2 bases at the 5′end of the miR-584-5p.

1. A method of increasing sensitivity of one or more medulloblastoma cancer cells to radiotherapy or chemotherapy, the method comprising:
(a) identifying a subject in need of treatment; and
(b) administering to the subject a therapeutically effective amount of a miR-584-5p before, during or after administration of radiotherapy or a chemotherapeutic agent, in an amount sufficient to increase sensitivity of one or more medulloblastoma cancer cells to the radiotherapy or a chemotherapeutic agent.

2. The method of claim 1, wherein the administration of the miR-584-5p renders the one or more medulloblastoma cancer cells susceptible to a cytotoxic dose of the radiotherapy or a chemotherapeutic agent.

3. The method of claim 1, the subject in need of treatment has been diagnosed with medulloblastoma prior to the administering step.

4. The method of claim 2, wherein the medulloblastoma is a wingless, Sonic Hedgehog, group 3 or group 4 subtype.

5. (canceled)

6. (canceled)

7. (canceled)

8. The method of claim 1, wherein the administration of the miR-584-5p renders the one or more medulloblastoma cancer cells susceptible to a cytotoxic dose of the radiotherapy or chemotherapeutic agent that is lower than the cytotoxic dose required in the absence of the miR-584-5p.

9. The method of claim 8, wherein the chemotherapeutic agent is vincristine.

10. The method of claim 1, wherein the miR-584-5p is administered systemically or locally.

11. The method of claim 1, wherein the miR-584-5p is encoded by a nucleic acid.

12. The method of claim 11, wherein the nucleic acid is located in a vector.

13. The method of claim 12, wherein the vector is a plasmid, cosmid, phagemid or a viral vector.

14. The method of claim 12, wherein the vector further comprises a lipid, lipid emulsion, liposome, nanoparticle or exosomes.

15. (canceled)

16. (canceled)

17. (canceled)

18. A synergistic composition for the treatment of cancer or tumor expressing increased levels of HDAC1 or eIF4E3, the composition comprising a miR-584-5p, and a microtubule-interfering chemotherapeutic agent.

19. The composition of claim 18, wherein the cancer is medulloblastoma.

20. The composition of claim 18, wherein the miR-584-5p is hsa-miR-584-5p comprising the nucleotide sequence set forth in SEQ ID NOs: 1 or 2.

21. The composition of claim 18, wherein the microtubule-interfering chemotherapeutic agent is vincristine, paclitaxel or docetaxel.

22.-39. (canceled)

40. A method of reducing growth of medulloblastoma cancer cells, the method comprising:
(a) administering a therapeutically effective amount of a composition comprising miR-584-5p to a subject having or suspected of having cancer cells reduced in expression of miR-584-5p; and
(b) administering to the subject a therapeutically effective amount of vincristine before, after or during administration of the composition comprising miR-584-5p, and wherein the medulloblastoma cancer cells have become sensitized to vincristine.

41. The method of claim 40, further comprising administering a radiotherapy, wherein the administration of a composition comprising miR-584-5p increases the sensitivity of the medulloblastoma cancer cells to the radiotherapy.

42. The method of claim 40, wherein the therapeutically effective amount of vincristine is lower than the therapeutically effective amount required in the absence of the composition comprising miR-584-5p.

43. The method of claim 40, further comprising administering radiotherapy, wherein the administration of a composition comprising miR-584-5p increases the sensitivity of the medulloblastoma cancer cells to both vincristine and radiotherapy.

44. The method claim 40, wherein the medulloblastoma cancer cells have reduced expression of miR-584-5p when compared to a reference sample before the administration of the composition comprising miR-584-5p.

45.-63. (canceled)


SEQUENCE LISTING (17 sequences)

SEQ ID NO: 1 - RNA, 22 nt, Artificial Sequence
  Synthetic construct
uuaugguuugccugggacugag

SEQ ID NO: 2 - RNA, 17 nt, Artificial Sequence
  Synthetic construct
uuaugguuugccuggga

SEQ ID NO: 3 - DNA, 22 bp, Artificial Sequence
  Synthetic construct
cccagggaaaagaaaaaccata

SEQ ID NO: 4 - DNA, 22 bp, Artificial Sequence
  Synthetic construct
cccagggaaaagaagggttgcg

SEQ ID NO: 5 - DNA, 23 bp, Artificial Sequence
  Synthetic construct
ccgttcttaactttgaaccataa

SEQ ID NO: 6 - DNA, 23 bp, Artificial Sequence
  Synthetic cosntruct
ccgttcttaactttgggttgcgg

SEQ ID NO: 7 - RNA, 7 nt, Artificial Sequence
  Synthetic construct
uaugguu

SEQ ID NO: 8 - DNA, 8 bp, Artificial Sequence
  Synthetic construct
gggttgcg

SEQ ID NO: 9 - DNA, 8 bp, Artificial Sequence
  Synthetic construct
ggttgcgg

SEQ ID NO: 10 - DNA, 50 bp, Artificial Sequence
  Synthtetic construct
tgtttttttctgtcccagggaaaagaaaaaccatacttaatgtgattttg

SEQ ID NO: 11 - DNA, 50 bp, Artificial Sequence
  Synthetic construct
tgtttttttctgtcccagggaaaagaagggttgcgcttaatgtgattttg

SEQ ID NO: 12 - DNA, 50 bp, Artificial Sequence
  Synthetic construct
tgtttttttctgtctcatagaaagaaagggttgcgcttaatgtgattttg

SEQ ID NO: 13 - DNA, 50 bp, Artificial Sequence
  Synthetic construct
gccacccattcttcccgttcttaactttgaaccataaagggtgccaggtc

SEQ ID NO: 14 - DNA, 51 bp, Artificial Sequence
  Synthetic construct
gccacccattcttcccgttcttaactttgggttgcggagggtgccaggtct

SEQ ID NO: 15 - DNA, 51 bp, Artificial Sequence
  Synthetic construct
gccacccattcttcctggtccgaacttgtggttgcggagggtgccaggtct

SEQ ID NO: 16 - DNA, 8 bp, Artificial Sequence
  Synthetic construct
aaccataa

SEQ ID NO: 17 - DNA, 8 bp, Artificial Sequence
  Synthetic construct
aaaccata